SEQUENCE LISTING (4 sequences)

SEQ ID NO: 1 - DNA, 24 bp, Artificial sequence
  primer_bind: Sense cox-2 primer
ggtctggtgcctggtctgatgatg

SEQ ID NO: 2 - DNA, 22 bp, Artificial sequence
  primer_bind: Antisense cox-2 primer
gtcctttcaaggagaatggtgc

SEQ ID NO: 3 - DNA, 25 bp, Artificial sequence
  primer_bind: GAPDH sense primer
caactacatggtttacatgttccaa

SEQ ID NO: 4 - DNA, 20 bp, Artificial sequence
  primer_bind: GAPDH antisense primer
gccagtggactccacgacgt

(19) United States
(12) Patent Application Publication
(10) Pub. No.: US 2002/0107219 A1
(43) Pub. Date: Aug. 8, 2002

(54) Adenoviral vector containing cyclooxygenase-2 promoter and uses thereof

(76) Inventors: David T. Curiel, Birmingham, AL (US); Masato Yamamoto, Birmingham, AL (US)

Correspondence Address: Benjamin Aaron Adler, ADLER & ASSOCIATES, 8011 Candle Lane, Houston, TX, 77071, US

(21) Appl. No.: 10/005,964

(22) Filed: Dec. 7, 2001

Related U.S. Application Data
(60) Provisional application No. 60/251,375, filed on Dec. 5, 2000.

(51) Int. Cl. A61K 48/00; C12N 15/861

(52) U.S. Cl. 514/44; 435/456; 435/320.1

(57) ABSTRACT

Cyclooxygenase-2 promoter is incorporated into adenoviral vector to mitigate the adverse effects of adenoviral suicide gene therapy by minimizing transgene expression in the liver. The promoters disclosed herein are promising tumor-specific promoters for adenoviral vector based gene therapy of gastrointestinal and pancreatic cancers.

### CROSS-REFERECNCE TO RELATED APPLICATION

This non-provisional patent application claims benefit of provisional patent application U.S. Serial No. 60/251,375, filed Dec. 5, 2000, now abandoned.

### FEDERAL FUNDING LEGEND

This invention was produced in part using funds obtained through grants from the National Institutes of Health, National Cancer Institute and United States Department of Defense. Consequently, the federal government has certain rights in this invention.

### BACKGROUND OF THE INVENTION

1. Field of the Invention

The present invention relates generally to adenoviral vectors and adenoviral gene therapy. More specifically, the present invention relates to an adenoviral vector containing cyclooxygenase-2 promoter and its application for the mitigation of toxicity in suicide gene therapy of gastrointestinal and pancreatic cancers.

2. Description of the Related Art

Advanced gastrointestinal cancers, especially those associated with distant metastatic disease, exhibit a very high mortality rate despite available therapies [1-2]. A variety of experimental therapeutic strategies have been explored for advanced gastrointestinal cancers including gene-based applications using suicide/toxin gene methods, such as herpes simplex virus thymidine kinase (HSV-tk) [3-5]. As adenoviral vectors can achieve efficient in situ gene delivery to tumors [6], they have been widely employed as a means to achieve the requisite intratumoral transgene expression for toxin gene approaches in locoregional gastrointestinal cancer models [7-8].

By contrast, derivation of adenoviral suicide gene therapy to the disseminated disease of gastrointestinal cancers has not been feasible currently. Because of the hepatotropism of adenovirus, systemically administered adenoviral vector localizes principally to the liver [9-10] and adenoviral suicide gene therapy of intrahepatic tumor leads to severe liver dysfunction [11-12]. Due to this vector physiology, the direct application of adenoviral vectors-based toxin gene approaches for disseminated or intrahepatic lesions has been hampered.

To overcome this obstacle, a number of strategies have been proposed to restrict the toxin gene expression to the tumor. The approach of transductional targeting seeks to alter vector tropism at the level of receptor interaction to achieve tumor-selective infection [13-14]. Alternatively, transcriptional targeting is based upon selective expression of toxin genes in tumor targets. Such an approach must thus employ a transcriptional control region with a selective “tumor on” phenotype. In addition, candidate promoters are also required to exhibit a “liver off” phenotype for mitigation of hepatotoxicity upon systemic delivery or the treatment of intrahepatic lesions.

Cyclooxygenase-2 (cox-2) is an inducible isoform of the cyclooxygenase family and is virtually undetectable in most tissues under physiological conditions [15-16]. On the other hand, cyclooxygenase-2 is closely linked to carcinogenesis and progression of colon cancers [17-18]; 85% of colon cancers, 45% of benign colon polyps and the majority of gastric cancers have been reported to show increased expression of cyclooxygenase-2 [17, 19, 26]. This tumor “on” liver “off” expression profile of cyclooxygenase-2 suggests the potential utility of this promoter for mitigating the toxicity caused by ectopic transgene expression.

The prior art is deficient in adenoviral vectors that are capable of mitigating liver toxicity in suicide gene therapy of gastrointestinal and pancreatic cancers. The present invention fulfills this long-standing need and desire in the art.

### SUMMARY OF THE INVENTION

The application of herpes simplex virus thymidine kinase-based adenoviral molecular chemotherapy for systemic malignant disease has been limited by ectopic expression of the transgene within the liver. The present invention demonstrated the cyclooxygenase-2 promoter as candidate transcriptional control region for gastrointestinal and pancreatic tumor targets. The properties of the cyclooxygenase-2 promoter in adenoviral vectors were analyzed in vitro and in vivo. These studies establish that the cyclooxygenase-2 promoter exhibits key properties for the feasibility of the adenoviral gene therapy for gastrointestinal and pancreatic cancers.

In one embodiment of the present invention, there is provided an adenoviral vector for the selective expression of a toxin gene in cancer cells. This adenoviral vector contains a toxin gene operably linked to a promoter of a gene with undetectable expression in liver; hence, the expression of the toxin gene is reduced in liver cells. In general, the toxin gene may be the herpes simplex virus thymidine kinase gene, the cytosine deaminase gene or the purine nucleoside phosphorylase gene. Preferably, the promoter is the cyclooxygenase-2 promoter, and the cancer cells are gastrointestinal cancer cells or pancreatic cancer cells.

In another embodiment of the present invention, there is provided a method of killing tumor cells with reduced liver toxicity in an individual by the adenoviral vector disclosed herein.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

The appended drawings have been included herein so that the above-recited features, advantages and objects of the invention will become clear and can be understood in detail. These drawings form a part of the specification. It is to be noted, however, that the appended drawings illustrate preferred embodiments of the invention and should not be considered to limit the scope of the invention.

FIG. 1 shows an analysis of cyclooxygenase-2 RNA. The RNA of the cell line used in these experiments was analyzed by reverse transcription followed by polymerase chain reaction. FIG. 1A shows the signal for cyclooxygenase-2 RNA detected at the position of 723 bp with cox-2 sense (5′ GGTCTGGTGCCTGGTCTGATGATG 3′, SEQ ID No. 1) and cox-2 antisense (5′GTCCTTTCAAGGAGAATGGTGC 3′, SEQ ID No. 2) primers. FIG. 1B shows glyceraldehyde 3-phosphate dehydrogenase (GAPDH) RNA was detected with using GAPDH sense (5′ CAACTACATGGTTTACATGTTCCAA 3′, SEQ ID No. 3) and GAPDH antisense (5′ GCCAGTGGACTCCACGACGT 3′, SEQ ID No. 4) primers.

FIG. 2 shows the structure of the recombinant adenoviral vectors. Adenoviral vectors were constructed by inserting the respective expression cassettes into the E1-deleted region of the E1- and E3-deleted backbone. Two different lengths of cox-2 promoters (cox-2 L: from −1432 to +59 and cox-2M: from −883 to +59) were placed 5′ of the transgene to drive expression. The luciferase expression vectors with cox-2L and cyclooxygenase-2 M promoters were named Adcox2L Luc and Adcox2M Luc, respectively. The HSV-tk expressing vectors with cox-2L and cox-2 M promoters were named Adcox2L TK and Adcox2M TK, respectively. The Simian virus 40 polyadenylation signal was placed on the 3′ of the transgene.

FIG. 3 shows the analysis of promoter property in various cell lines. Each cell line was infected with cox-2 promoter driven luciferase expression vectors and luciferase activity was analyzed. The results were shown after standardization with protein concentrations. FIGS. 3A and 3B are the results with cyclooxygenase-2 M and L promoter driven regular adenoviral vectors, respectively. FIGS. 3C and 3D are those with the RGD modification.

FIG. 4 illustrates the cyclooxygenase-2 promoter activity in major organs. Luciferase activities were analyzed in vivo in liver (FIG. 4A), spleen (FIG. 4B), lung (FIG. 4C) and kidney (FIG. 4D) after tail vein administration of cyclooxygenase-2 or CMV promoter driven luciferase expression vectors. The luciferase activities are shown as RLU per mg protein.

FIG. 5 shows the cyclooxygenase-2 promoter activity in subcutaneous tumors. The luciferase activities were analyzed in three subcutaneous tumors after intratumoral injection of cyclooxygenase-2 or CMV promoter driven luciferase expression vectors. The luciferase activities are shown as RLU per mg protein. FIG. 5A: MKN28; FIG. 5B: MKN45 and FIG. 5C: LS174T.

FIG. 6 shows the cytocidal effect of the cyclooxygenase-2 promoter driven HSV-tk expression vectors. Cells were infected with cyclooxygenase-2 promoter driven HSV-tk expressing vectors (Adcox2M TK and Adcox2M TK) and the number of surviving cells was analyzed using the MTS method after 5 days of treatment with various concentrations of GCV. AdCMV TK and AdGFP were used as positive and negative control vectors, respectively. FIG. 6A: MKN28 cells, cyclooxygenase-2 positive; FIG. 6B: MKN45 cells, cyclooxygenase-2 positive; FIG. 6C: LS174T cells, cyclooxygenase-2 positive and FIG. 6D: KATO3 cells, cyclooxygenase-2 negative.

FIG. 7 shows the cytocidal effect of the cyclooxygenase-2 promoter driven HSV-tk expression vectors in vivo. Cox-2 positive MKN45 cells were inoculated subcutaneously into Nur/Nu mice. When the tumor grew to 6-8 mm, 10⁹ pfu of Adcox-2LTK (circle), AdCMVTK (triangle) or AdCMVLuc (square) were injected into the tumor (day 0). From day 1 to day 14, 50 mg/kg of ganciclivir was administered intraperitoneally twice a day. In the group treated with AdCMVTK, more than half of the mice died before day 14. There was no death in other groups. The tumor volume of mice treated with Adcox-2TK was significantly smaller than that in mice treated with AdCMVLuc (p<0.01). AdCMVTK indicated therapeutic effect but it was not statistically significant due to least number of points (*).

FIG. 8 shows the mitigation of toxicity of suicide gene therapy with the cyclooxygenase-2 L promoter. After systemic administration of HSV-tk expressing adenoviral vector (10⁹ pfu) followed by 5 days of GCV (50 mg/kg weight, twice a day, intraperitoneal), the major organs were macroscopically observed. FIG. 8A: AdCMV TK with GCV; FIG. 8B: Adcox2L TK with GCV; FIG. 8C: AdCMV TK without GCV and FIG. 8D: no treatment.

FIG. 9 shows the mitigation of toxicity of suicide gene therapy with cyclooxygenase-2 L promoter. The major organs of the mice in each group were microscopically analyzed after H and E staining (original magnification×600). The findings of the liver are shown in this figure. The open arrows indicate cells undergoing individual necrosis. Solid arrow indicates mitosis. FIG. 9A: AdCMV TK with GCV; FIG. 9B: Adcox2L TK with GCV; FIG. 9C: AdCMV TK without GCV and FIG. 9D: no treatment.

FIG. 10 shows blood sample analysis that shows mitigation of toxicity of suicide gene therapy with the cyclooxygenase-2 L promoter. The blood samples of each group were analyzed for ALT, AST, total bilirubin and LDH. Group A: AdCMV TK with GCV; group B: Adcox2L TK with GCV; group C: no virus with GCV; group D: AdCMV TK without GCV; group E: Adcox2L TK without GCV; group F: no treatment.

FIG. 11 shows tumor-specificity of cyclooxygenase-2 promoter in human pancreatic carcinoma cells. Primary human pancreatic carcinoma cells: p6.3 (FIG. 11A) and p10.5 (FIG. 11B) and established human pancreatic carcinoma cell lines: BxPC-3 (FIG. 11C), Capan-1 (FIG. 11D), Hs 766-T (FIG. 11E) and MIA PaCa-2 (FIG. 11F) were infected with Ad vectors expressing the luciferase gene under control of two regions of the cyclooxygenase-2 promoter: COX-2M (−883 bp—−+59 bp; AdCOX-2M Luc) and COX-2L(−1432 bp—−+59 bp; AdCOX-2L Luc) or the CMV promoter (AdCMVLuc) at an MOI of 1, 10 and 100. After incubation at 37° C. for 48 h, the cells were lysed, the protein concentration of the lysates was determined, and the RLU of luciferase/milligram of total cellular protein is shown graphically. The results are the mean of duplicate assays. Each point represents the mean and the standard deviation of the two determinations.

FIG. 12 shows GCV sensitivity of pancreatic carcinoma cell lines transduced with Adcox-2M Tk, AdCOX-2L Tk or AdCMV Tk. Established human pancreatic carcinoma cell lines: BxPC-3 (FIG. 12A), Capan-1 (FIG. 12B), Hs 766-T (FIG. 12C), MIA PaCa-2 (FIG. 12D) and the human gastric cancer cell line KATO III (FIG. 12E) were infected with AdCOX-2M Tk, AdCOX-2L Tk, AdCMV Tk or AdCMVGFP at an MOI of 500 for 5 hours. GCV w as applied at the concentration ranging from 0 to 10⁴ μM and 5 days later the percentage of surviving cells was calculated from the number of surviving cells without GCV (=100%) and the number of surviving cells at various concentration of GCV. The number of surviving cells was determined by an MTT assay. The results are the mean of triplicate assays.

### DETAILED DESCRIPTION OF THE INVENTION

The following abbreviations may used herein: ALT, alanine aminotransferase; AST, aspartate aminotransferase; CMV, cytomegalovirus; cox-2, cyclooxygenase-2; GAPDH, Glyceraldehyde 3-phosphate dehydrogenase; GCV, ganciclovir; HSV-tk, herpes simplex virus thymidine kinase; LDH, lactate dehydrogenase; MOI, multiplicity of infection; pfu, plaque forming unit; RLU, relative light unit; RT-PCR, reverse transcription -and polymerase chain reaction.

Adenoviral suicide gene therapy to the disseminated disease of gastrointestinal cancers has not currently been feasible due to liver toxicity. The present invention demonstrates that one can mitigate the toxicity of this treatment using the promoters of cyclooxygenase-2, whose expression are virtually undetectable in liver but is detected in many gastrointestinal and pancreatic cancers.

Two different lengths of cyclooxygenase-2 promoters were incorporated into adenoviral vectors. The promoters were characterized with luciferase expression vectors. The specific cytocidal effect and in vivo toxicity were analyzed with thymidine kinase expression vectors. The specificity of the cyclooxygenase-2 promoter was well preserved in the adenoviral vector In vivo, the cox-2 promoter cox-2L (−1432/+59) showed very little activity in the liver but accomplished high activity in cyclooxygenase-2 positive subcutaneous tumors comparable to the cytomegalovirus promoter. The cyclooxygenase-2 promoter driven thymidine kinase-expressing vectors showed a cytocidal effect specifically in cyclooxygenase-2 positive cells. When mice were treated with the thymidine kinase-expressing vector and ganciclovir, the cyclooxygenase-2 promoter successfully mitigated the fatal hepatotoxicity which was observed with the cytomegalovirus-promoter driven vector. Thus, the cyclooxygenase-2 promoter successfully mitigated the adverse effects of adenoviral suicide gene therapy by minimizing transgene expression in the liver.

Tumor-specificity of the cyclooxygenase-2 promoters in human pancreatic cancer was also investigated with the aim to improve the selectivity of therapeutic gene expression. Recombinant adenoviral vectors containing either the luciferase (Luc) reporter gene under the control of the cyclooxygenase-2 or the herpes simplex virus thymidine kinase (Tk) gene under the control of the cyclooxygenase-2 promoter were compared with the expression driven by the cytomegalovirus (CMV) promoter. Of the two cyclooxygenase-2 promoter regions (COX-2M and COX-2L), both revealed a high activity in primary pancreatic carcinoma cells, while in the established pancreatic carcinoma cell lines COX-2L has an approximately equal high activity as compared to CMV. In addition, both AdCOX-2M Tk and AdCOX-2L Tk induced marked cell death in response to ganciclovir (GCV) in three out of four established pancreatic carcinoma cell lines. From these results, it is concluded that the cyclooxygenase-2 promoters are promising tumor-specific promoters for adenoviral vector based gene therapy of pancreatic cancer.

As used herein, a “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains (consensus sequences) responsible for the binding of RNA polymerase. Eukaryotic promoters often, but not always, contain “TATA” boxes and “CAT” boxes.

A coding sequence is “operably linked” and “under the control” of a promoter sequence in a cell if the promoter sequence effectively controls the transcription of the coding sequence.

A “heterologous DNA sequence” within a larger DNA molecule is an identifiable segment of DNA that is not found in association with the larger DNA molecule in nature.

The term “CAR-independent infectivity” refers to the entry of adenovirus into a cell by receptors different from the coxsackie-adenovirus receptor (CAR).

The term “RGD-integrin interaction” refers to the arginine-glycine-aspartic acid (RGD) residues in a peptide binding to the integrin receptor molecules.

The term “replication-competent adenoviruses” refers to an adenovirus capable of replication (i.e., an adenovirus that yields progeny).

It is specifically contemplated that pharmaceutical compositions may be prepared using the novel adenovirus of the present invention. In such a case, the pharmaceutical composition comprises the novel adenovirus of the present invention and a pharmaceutically acceptable carrier. A person having ordinary skill in this art would readily be able to determine, without undue experimentation, the appropriate dosages and routes of administration of this adenovirus of the present invention. When used in vivo for therapy, the adenovirus of the present invention is administered to the patient or an animal in therapeutically effective amounts, i.e., amounts that eliminate or reduce the tumor burden. It may be administered parenterally, preferably intravenously, but other routes of administration will be used as appropriate. The dose and dosage regimen will depend upon the nature of the cancer (primary or metastatic) and its population, the patient, the patient's history and other factors. The amount of adenovirus administered will typically be in the range of about 10¹⁰ to about 10¹¹ viral particles per patient. The schedule will be continued to optimize effectiveness while balanced against negative effects of treatment. See Remington's Pharmaceutical Science, 17th Ed. (1990) Mark Publishing Co., Easton, Pa.; and Goodman and Gilman's: The Pharmacological Basis of Therapeutics 8th Ed (1990) Pergamon Press; which are incorporated herein by reference. For parenteral administration, the adenovirus will most typically be formulated in a unit dosage injectable form (solution, suspension, emulsion) in association with a pharmaceutically acceptable parenteral vehicle. Such vehicles are preferably non-toxic and non-therapeutic. Examples of such vehicles are water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives.

In one embodiment of the present invention, there is provided an adenoviral vector for the selective expression of toxin gene in cancer cell. The adenoviral vector contains a toxin gene operably linked to a promoter of a gene with undetectable expression in liver; hence, the expression of the toxin gene is reduced in liver cells. In general, the toxin gene is the herpes simplex virus thymidine kinase gene, the cytosine deaminase gene or the purine nucleoside phosphorylase gene. Furthermore, the adenoviral vector contains a RGD motif in the HI loop of the adenovirus fiber protein. Preferably, the promoter is cyclooxygenase-2 promoter, and the cancer cells are gastrointestinal cancer cells or pancreatic cancer cells.

In conjunction with the above-mentioned adenoviral vector, a method of killing tumor cells with reduced liver toxicity in an individual is disclosed herein, comprising the steps of: administering to the individual an effective amount of an adenoviral vector comprising of a toxin gene operably linked to a promoter of a gene with undetectable expression in liver, wherein expression of said toxin gene is reduced in liver cells and expression of said toxin gene in tumor cells results in killing of said tumor cells. The adenoviral vector can further contain a RGD motif in the HI loop of the adenovirus fiber protein. In general, the toxin gene is the herpes simplex virus thymidine kinase gene, the cytosine deaminase gene o r the purine nucleoside phosphorylase gene. When the toxin gene carried by the adenovirus is the herpes simplex virus thymidine kinase gene, the present method further includes a step of administering ganciclovir to the individual. Preferably, the promoter is cyclooxygenase-2 promoter, and the cancer cells are gastrointestinal cancer cells or pancreatic cancer cells.

The following examples are given for the purpose of illustrating various embodiments of the invention and are not meant to limit the present invention in any fashion:

### EXAMPLE 1

### Cell Culture and Animals

The Caco-2, WiDr and LS174T colon cancer cell lines (American Type Culture Collection, ATCC, HTB-37, CCL-218 and CL 188, respectively; Manassas, Va.) were grown in Minimum Essential Medium with non-essential amino acids (Mediatech, Herndon, Va.). The MKN28, MKN45 and KATO-3 gastric cancer cell lines (Japanese Collection of Research Bioresources, JCRB, JCRB0253, JCRB0254, JCRB0611, respectively; Tokyo, Japan) and lung cancer cell lines A549 (ATCC CCL-185) and H358 (ATCC CRL-5807) were grown in RPMI 1640 (Mediatech). The HepG2 (ATCC HB-8065), HuH-6 clone5 (JCRB0401), HuH-7 (JCRB0403) liver cancer cell lines and 293 adenoviral transformed human embryonic kidney cell line (ATCC CRL-1573) were maintained with Dulbecco's -Modified Eagles Medium. All the media were supplemented with 10% fetal calf serum, penicillin (100 IU/ml) and streptomycin (100 μg/ml). Cells were incubated at 37° C. and 5% CO₂.

Female C57BL/6 mice (Charles River, Wilmington, Mass.) and female Nu/Nu athymic nude mice (Frederick Cancer Research, Frederick, Md.) (6-8 week old) were used for in vivo experiments. All animals received humane care based on guidelines set by the American Veterinary Association. All the experimental protocols involving live animals were reviewed and approved by the Institutional Animal Care and Use Committee of the University of Alabama at Birmingham.

### EXAMPLE 2

### Analysis Of Cox-2 RNA Status

The cyclooxygenase-2 RNA status of cell lines was analyzed by reverse transcription and polymerase chain reaction (RT-PCR). Total RNA was extracted from semi-confluent cell cultures on 10 cm dishes using the RNeasy mini RNA extraction kit (QIAGEN, Valencia, Calif.) as described by the manufacturer. Five hundred nanograms of total RNA were reverse transcribed with oligo-dT primer and amplified by polymerase chain reaction using the GeneAMP RNA PCR Kit (Perkin Elmer, Branchburg, N.J.) as described by the manufacturer except the extension time was 60 sec. The primers used for the analysis of cyclooxygenase-2 RNA were cyclooxygenase-2 sense (5′ GGTCTGGTGCCTGGTCTGATGATG 3′, SEQ ID No. 1) and cyclooxygenase-2 antisense (5′ GTCCTTTCAAGGAGAATGGTGC 3′, SEQ ID No. 2). As a control, Glyceraldehyde 3-phosphate dehydrogenase (GAPDH) RNA was also analyzed in the same way using GAPDH sense (5′ CAACTACATGGTTTACATGTTCCAA 3′, SEQ ID No. 3) and GAPDH antisense (5′ GCCAGTGGACTCCACGACGT 3′, SEQ ID No. 4) primers.

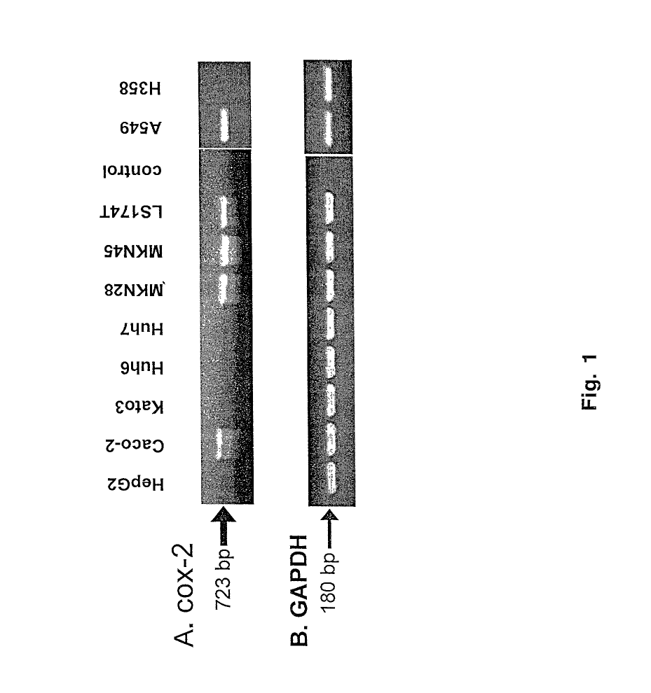

The cyclooxygenase-2 RNA status of the cell lines used for this experiment was shown in FIG. 1. Because the primers were designed to have three introns between them, the signal detected at 723 bp represented complementary DNA that was reverse transcribed from RNA. In the liver cell lines (HuH6, Huh7 and HepG2), there was no band detected. Two gastric cancer cell lines (MKN28 and MKN45) and one lung cancer cell line (A549) showed a very strong signal and two colon cancer cell lines (LS174T and Caco-2) showed a moderate signal. On the other hand, one gastric cancer cell line (KATO-3) and a lung cancer cell line (H358) did not show a signal for cox-2 RNA.

### EXAMPLE 3

### Adenovirus Vectors Containing the Cox-2 Promoter

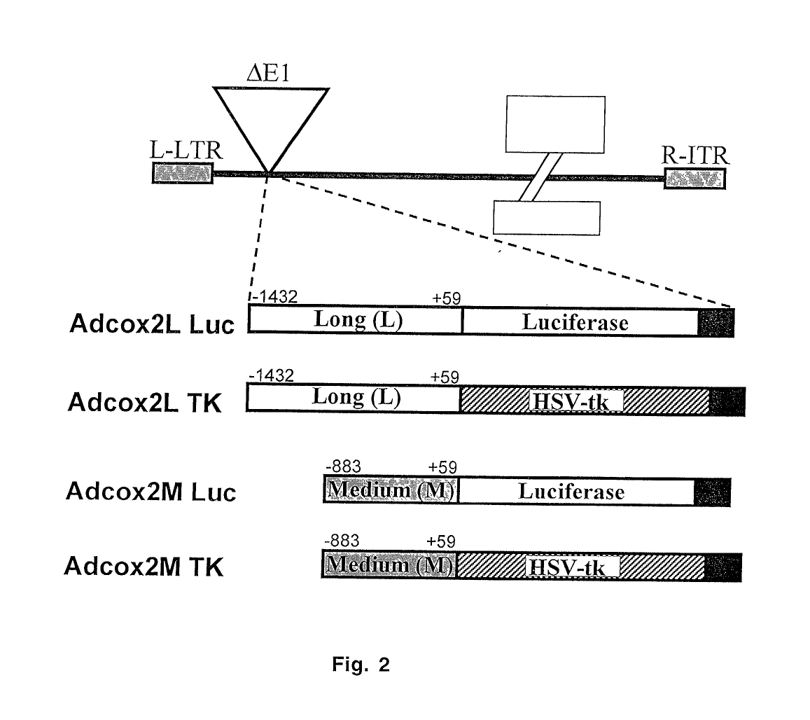

The recombinant adenoviral vectors that express firefly luciferase and HSV-tk respectively were constructed through homologous recombination in Escherichia coli using the AdEasy system [27] (FIG. 2). All vectors used in these experiments had the transgene cassettes in the E1 deleted region of adenoviral vector backbone. Two different lengths of promoters derived from phPES2 id (provided by Drs. Inoue and Tanabe at National Cardiovascular Center Research Institute, Japan [28-29]) were placed in front of each transgene for selective expression: one is cox-2L (−1432/+59, the whole 5′ control region of phPES2) and the other is cox-2M (−883/+59, SacI-HindIII fragment of cox-2L). Though three major control regions of the cyclooxygenase-2 promoter (binding sites of NF-KB, NFIL-6 and CRE) exist within 300 base pairs from transcription initiation site, the longer control regions were used to achieve as much fidelity as possible. The resultant vectors expressing luciferase were designated Adcox-2L Luc and Adcox-2M Luc, and those expressing HSV-tk were designated Adcox-2L TK and Adcox-2M TK, respectively. The luciferase gene and Simian virus 40 poly adenylation signal were from pGL3 Basic (Promega, Madison Wis.). As control vectors that expressed the transgene ubiquitously, luciferase and HSV-tk expression vectors with the cytomegalovirus (CMV) immediate early promoter (derived from plasmid pCEP4, Invitrogen, Carlsbad, Calif.) instead of the cyclooxygenase-2promoter were also constructed and named AdCMV Luc and AdCMV TK respectively.

The infectivity enhanced version of luciferase expression vectors, RGDcox-2L Luc and RGDcox-2M Luc, were constructed with cox-2L and cox-2M promoters respectively and plasmid pVK503C [31]. These vectors confer coxsackie-adenovirus receptor independent infection via the integrin binding of the RGD motif inserted into the adenovirus fiber HI-loop [30-31].

The viruses were propagated in the adenovirus packaging cell line, 293, and purified by double CsCl density gradient centrifugation, followed by dialysis against phosphate-buffered saline with 10% glycerol. The vectors were titrated by plaque assay and stocked at −80° C. until usage.

### EXAMPLE 4

### In Vitro Analysis Of The Cox-2 Promoter In An Adenoviral Construct

The activity of the cyclooxygenase-2 promoter in an adenovirus context was analyzed by infection of cells with luciferase expression vectors. One day after plating 50,000 cells per well on 24 well plate, cells were infected at a multiplicity of infection (MOI) of 50 plaque forming unit (pfu) per cell with Adcox2L Luc and Adcox2M Luc, respectively, in Dulbecco's Modified Eagles Medium with 5% fetal calf serum (infection medium) for one hour. Two hours later, the infection medium was replaced with the appropriate complete medium. After 48 hours of cultivation, the cells were lysed with Cell Culture Lysis Buffer (Promega) and resultant lysates were analyzed with the Luciferase Assay System (Promega). The protein concentration was determined with the DC protein assay (Bio-Rad, Hercules, Calif.). For RGD-containing vectors, the same experiments were performed using RGDcox-2L Luc and RGDcox-2M Luc at an MOI of 10 pfu per cell. All the experiments were done in triplicate.

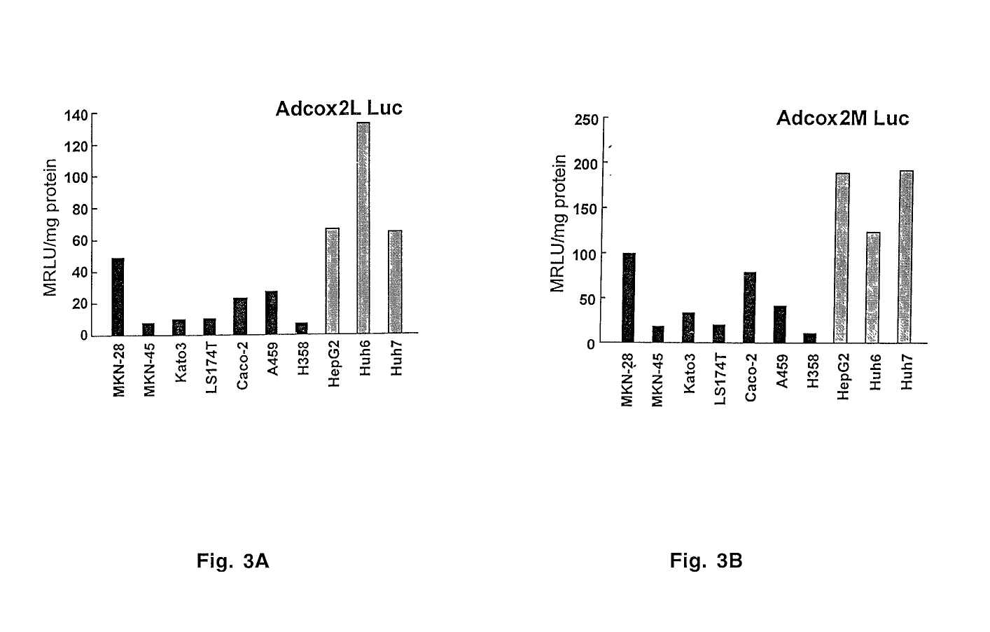

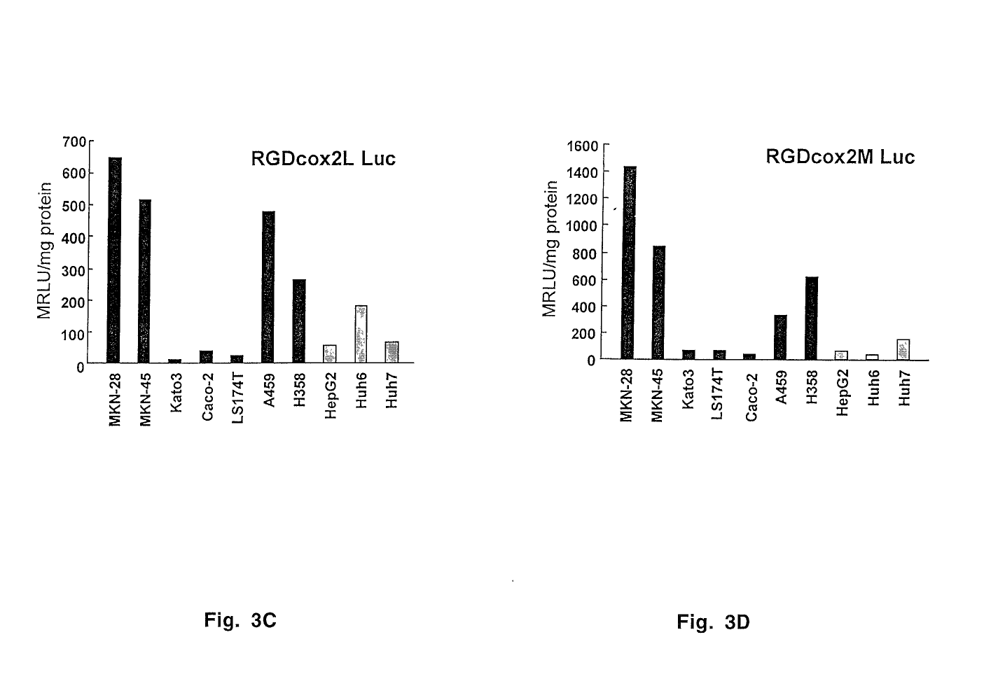

As shown in FIG. 3, the cox-2 positive cells expressed luciferase but cells that were considered to be negative for cox-2 expression in RT-PCR analysis also showed high luciferase activity. This was most remarkable in liver derived cells (FIG. 3A and B). In case of using the vectors with RGD modification (RGDcox2L Luc and RGDcox2M Luc), which can infect cells in a coxsackie-adenovirus receptor-independent manner, the expression in the non-liver derived cells increased and, as a result, the relative expression in the liver derived cells became much lower than that in most of the cox-2 positive cell lines (FIG. 3C and D). In comparison to the cyclooxygenase-2 L promoter, the cyclooxygenase-2 M promoter showed slightly higher activity, but the overall profiles of these two were almost similar.

### EXAMPLE 5

### In Vivo Analysis Of The Cox-2 Promoter In An Adenoviral Construct: Activity In Major Organs

To analyze the luciferase gene expression in mouse organs, C57BL/6 mice received 10⁹ pfu of Adcox2L Luc, Adcox-2M Luc or AdCMV Luc intravenously via the tail vein. Two days later, the livers, lungs, kidneys and spleens were harvested to measure luciferase expression.

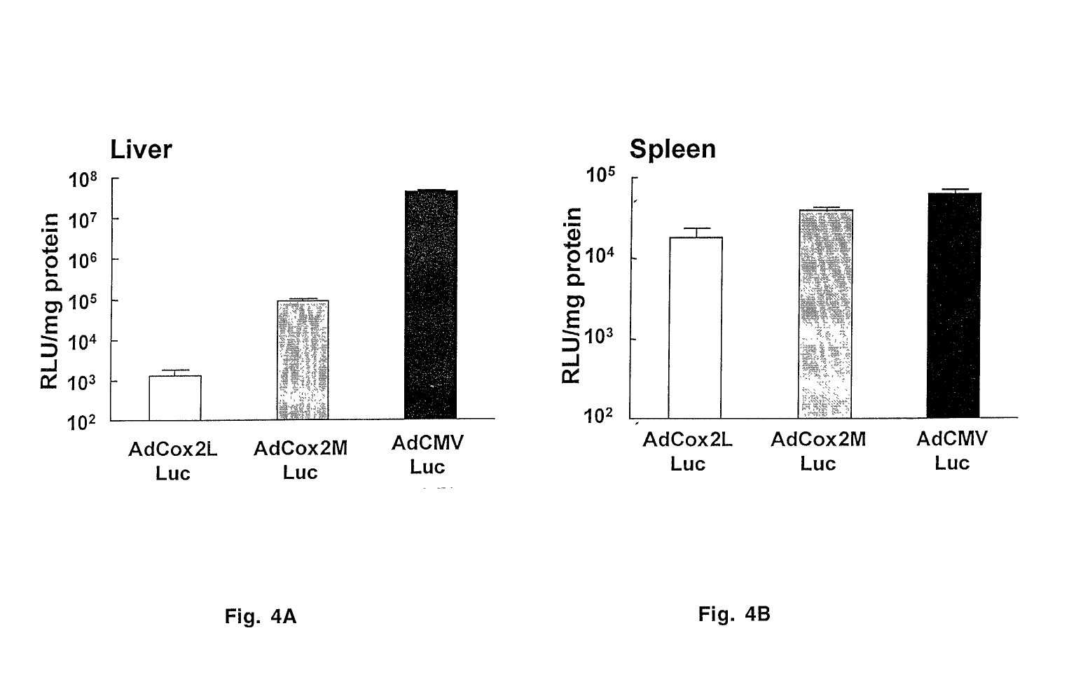

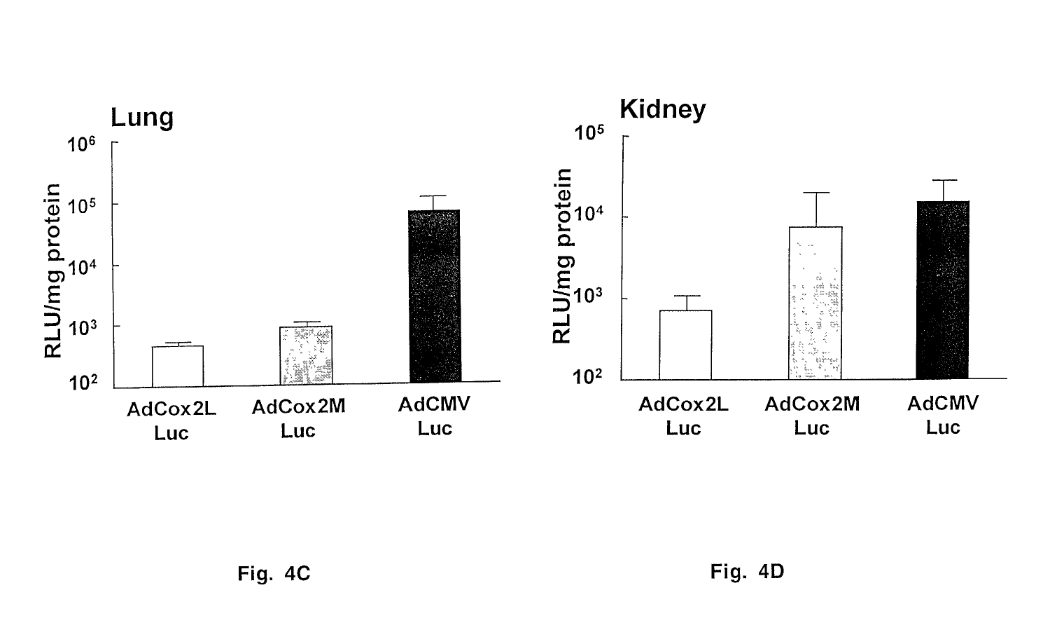

After intravenous administration of Adcox2L Luc, Adcox2M Luc and AdCMV Luc, the activity of the cox-2 promoter in four major organs (liver, lung, kidney and spleen) was assessed by luminometric analysis (FIG. 4). In the liver, the luciferase activity with Adcox2M Luc (89500 relative light units (RLU)/mg protein, n=3) was less than 1/400 of that with AdCMV Luc (41700000 RLU/mg protein, n=3). The activity with Adcox2L Luc, which had longer promoter sequence, (1320 RLU/mg protein, n=3) was even lower than that with Adcox2M Luc and less than 1/30000 of AdCMV Luc (FIG. 4A).

In contrast, the activities in the spleen with these three vectors were within the same order of magnitude (16200, 39300 and 61700 RLU/mg protein for Adcox2L Luc, Adcox2M Luc and AdCMV Luc, respectively, n=3) (FIG. 4B). However, in the lung, each of the two vectors with cyclooxygenase-2 promoters showed two orders of magnitude lower activity than that seen with CMV promoter (FIG. 4C). In the kidney, only Adcox2L Luc showed lower activity than that seen with others (FIG. 4D).

### EXAMPLE 6

### In Vivo Analysis Of Cox-2 Promoter In An Adenoviral Construct: Activity In Subcutaneous Tumors

For the analysis of subcutaneous tumors, 2×10⁷ cultivated cells were inoculated subcutaneously into the flank of the congenitally athymic nude mice, and 10⁹ pfu of Adcox2L Luc, Adcox2M Luc or AdCMV Luc were injected into the tumors when tumors of 6-8 mm diameter were formed. Two days later, the tumors were resected for luciferase analysis. All the organs and tumors were rapidly frozen on dry ice and stored at −80° C. until assayed. On the day of analysis, tissues were ground into fine powder with a pestle and mortar in an ethanol/dry ice bath. The tissue powders were lysed with Cell Culture Lysis Buffer (Promega) and, after three rounds of freezing and thawing followed by centrifugation, the recovered supernatants were analyzed for luciferase activity with the Luciferase Assay System (Promega). The protein concentration was determined with DC protein assay (Bio-Rad). All the experiments were done in triplicate.

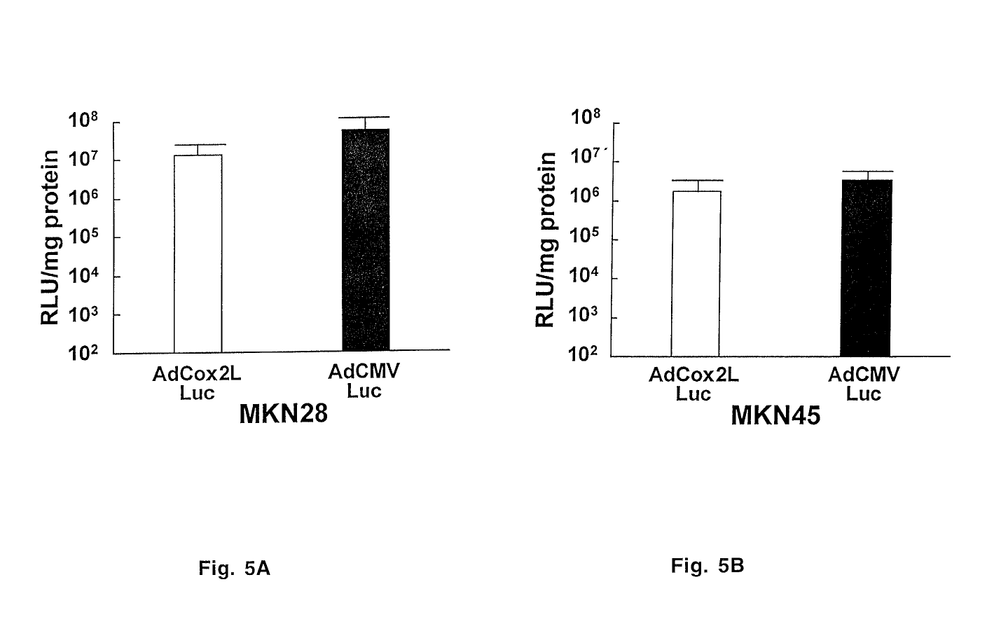

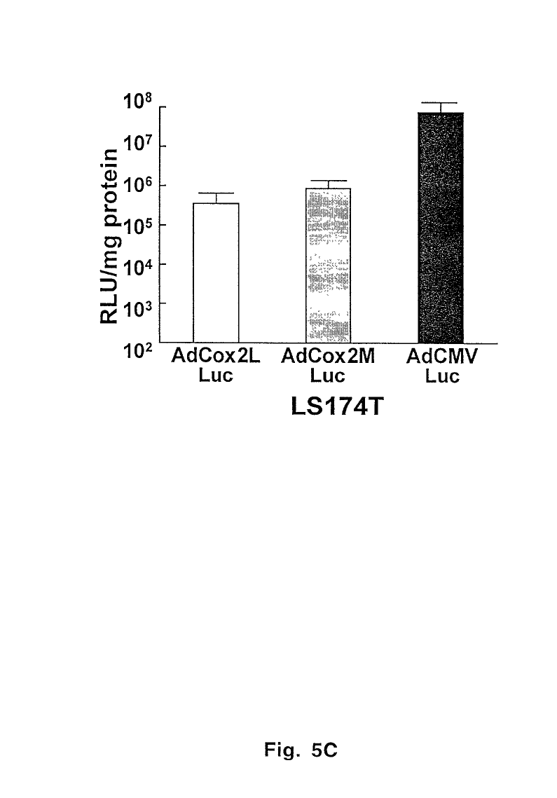

The activity of the cyclooxygenase-2 promoter in an adenoviral construct was compared with the CMV promoter in gastrointestinal cancer subcutaneous tumors. In subcutaneous tumor xenografts of two cyclooxygenase-2 strongly positive cell lines, MKN28 and MKN45, the activities of the cox-2L promoter were comparable to the CMV promoter (12900000 RLU/mg protein to 55100000 RLU/mg protein for MKN28, 1760000 RLU/mg protein to 3360000 RLU/mg protein for MKN45) (FIG. 5A and 5B). In the case of a moderately cyclooxygenase-2 positive cell line, LS174T, the activity of the cox-2 promoter was detected but lower than that with CMV promoter (362000, 868000 and 73900000 RLU/mg protein with Adcox2L Luc, Adcox2M Luc and AdCMVLuc, respectively) (FIG. 5C). Thus, adenoviral vector mediated delivery to subcutaneous tumors in vivo could accomplish strong transgene expression in cyclooxygenase-2 positive cells at the level comparable to the CMV promoter.

### EXAMPLE 7

### Cytocidal Effect Of The Cox-2 Promoter Driven HSV-Tk Expression Vector In Combination With GCV

Tumor cells were plated in 96 well plates at a density of 3,000 per well. The following day, the cells were infected for 5 hours with 100 μl of infection medium containing Adcox2L TK, Adcox2M TK and AdCMV TK, respectively. To compensate the relatively large infection volume, the infections were performed a t MOI of 500. The infection mediums were then replaced with appropriate medium supplemented with various concentrations of ganciclovir (GCV). As a negative control, a green fluorescent protein expression vector (AdGFP) was used. After 5 days of incubation, the number of surviving cells was analyzed by the MTS method using the Cell Titer 96 Aqueous Non-Radioactive Cell Proliferation Assay (Promega) and an automated E max spectrophotometric plate reader (Molecular Device Corp., Sunnyvale, Calif.) as described by the manufacturers. All the experiments were done in triplicate.

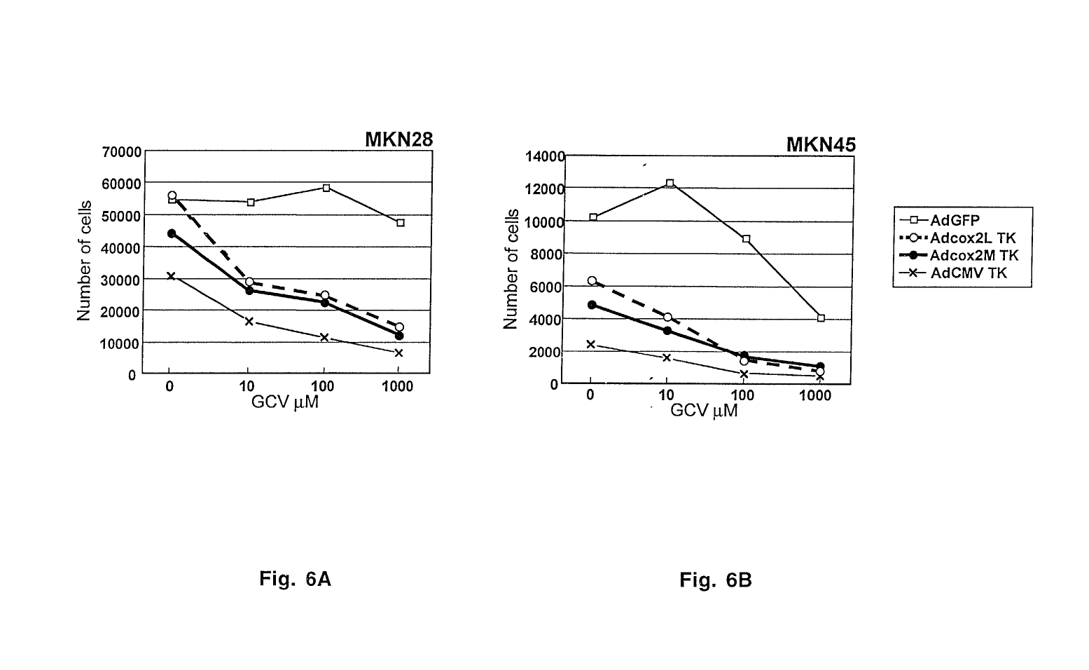

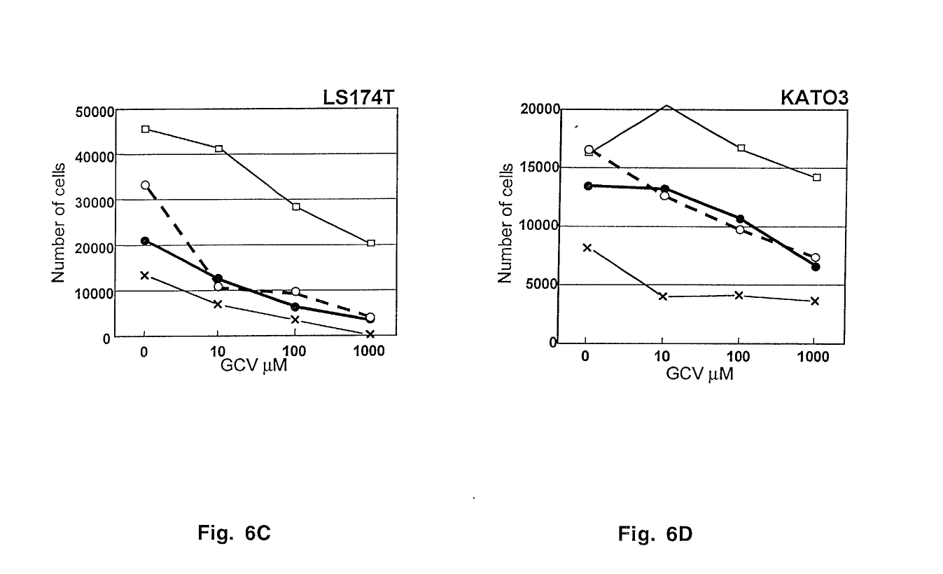

The specificity of the cytocidal effects with the cyclooxygenase-2 promoter driven HSV-tk expression vectors was analyzed in cell lines. An obvious cytocidal effect comparable to AdCMV TK was observed with Adcox2M TK and Adcox2L TK not only in two cyclooxygenase-2 strongly positive cells (MKN28 and MKN45, FIG. 6A and 6B) but also in the cyclooxygenase-2 moderately positive cell line (LS174T, FIG. 6C). In contrast, in cyclooxygenase-2 negative cell line (Kato-3), a strong cytocidal effect was observed only with AdCMV TK (FIG. 6D). These results clearly indicate that either length of cyclooxygenase-2 promoter (cyclooxygenase-2 L and cyclooxygenase-2 M) conferred selective cell killing in cyclooxygenase-2 positive cancer cells in the HSV-tk expressing adenoviral vector.

The cytocidal effects of the cyclooxygenase-2 promoter driven HSV-tk expression vectors were further confirmed by in vivo studies. Cyclooxygenase-2 positive MKN45 cells were inoculated subcutaneously into Nur/Nu mice. When the tumor grew to 6-8 mm, 10⁹ pfu of Adcox-2LTK, AdCMVTK or AdCMVLuc were injected into the tumor (day 0). From day 1 to day 14, 50 mg/kg of ganciclivir was administered intraperitoneally twice a day.

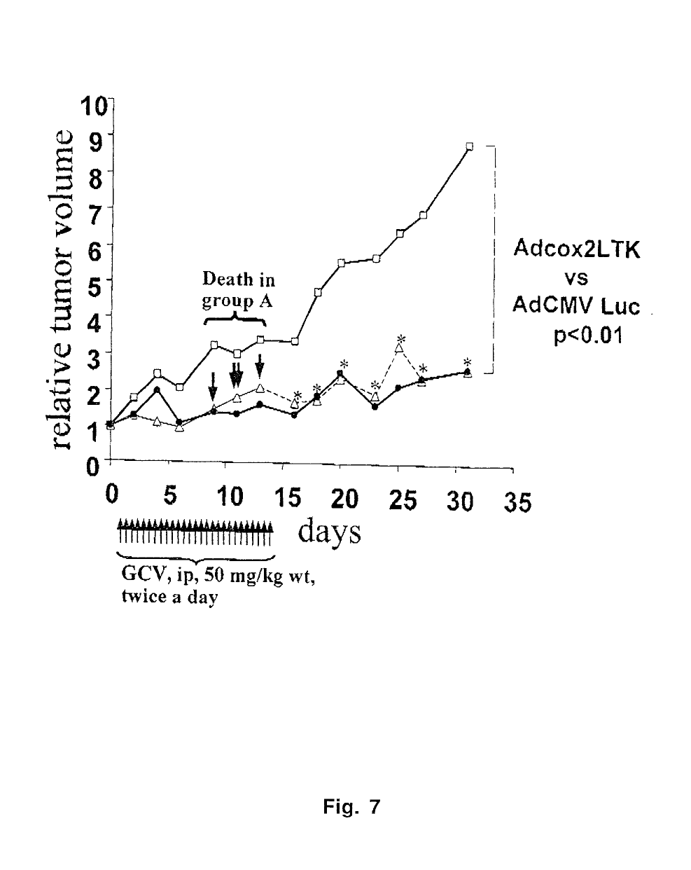

As shown in FIG. 7, the tumor volume of mice treated with Adcox-2TK was significantly smaller than that in mice treated with AdCMVLuc. thus indicating that the cyclooxygenase-2 promoter driven HSV-tk expression vectors can effectively kill cyclooxygenase-2 positive cancer cells in the context of TK-GCV gene therapy approach.

### EXAMPLE 8

### Mitigation Of Hepatotoxicity Of Adenoviral Suicide Gene Therapy Employing The Cox-2 Promoter

To investigate the toxicity induced by the HSV-tk/GCV system, each group of ten C57BL/6 mice received a tail vein injection of AdCMV TK (10⁹ pfu), Adcox2LTK (10⁹ pfu) or phosphate-buffered saline on day 0. Five of each group received GCV (50 mg/kg body weight) intraperitoneally twice daily from day 1 to day 5 and the others did not. The mice were sacrificed on day 6 and the blood samples were collected to analyze total bilirubin, alanine aminotransferase (ALT), aspartate aminotransferase (AST) an d lactate dehydrogenase (LDH) at a veterinary laboratory (ANTECH Diagnostics, Farmingdale, N.Y.). For histopathological analysis, the major organs were fixed with 10% buffered formaldehyde, paraffin-embedded and cut in to 4 μm section, followed by deparaffinization and staining with hematoxylin and eosin.

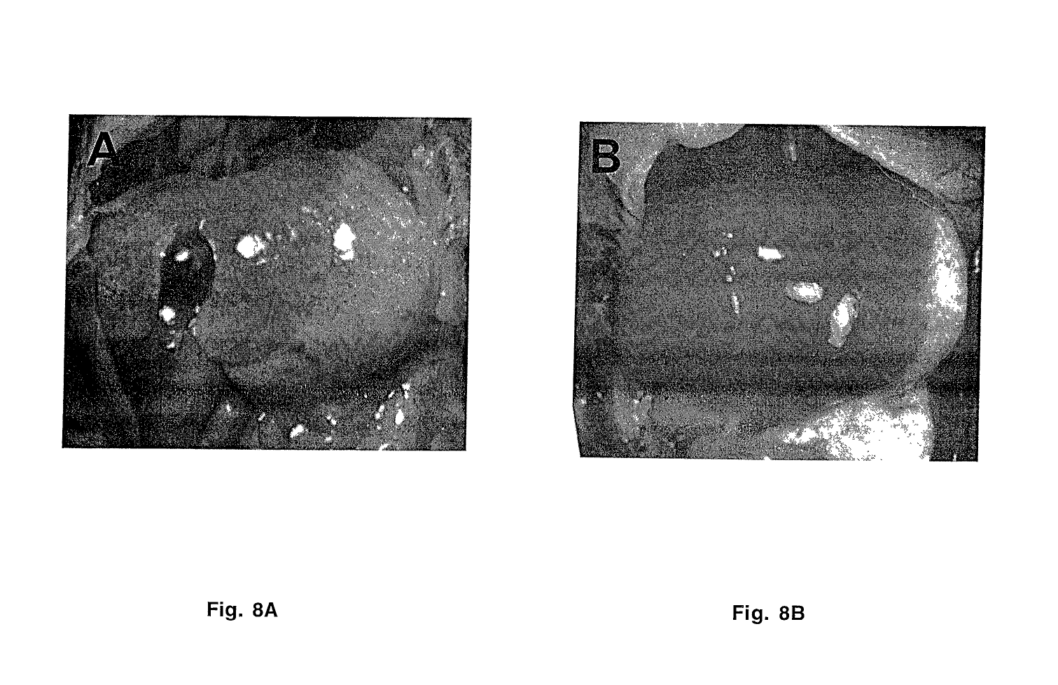

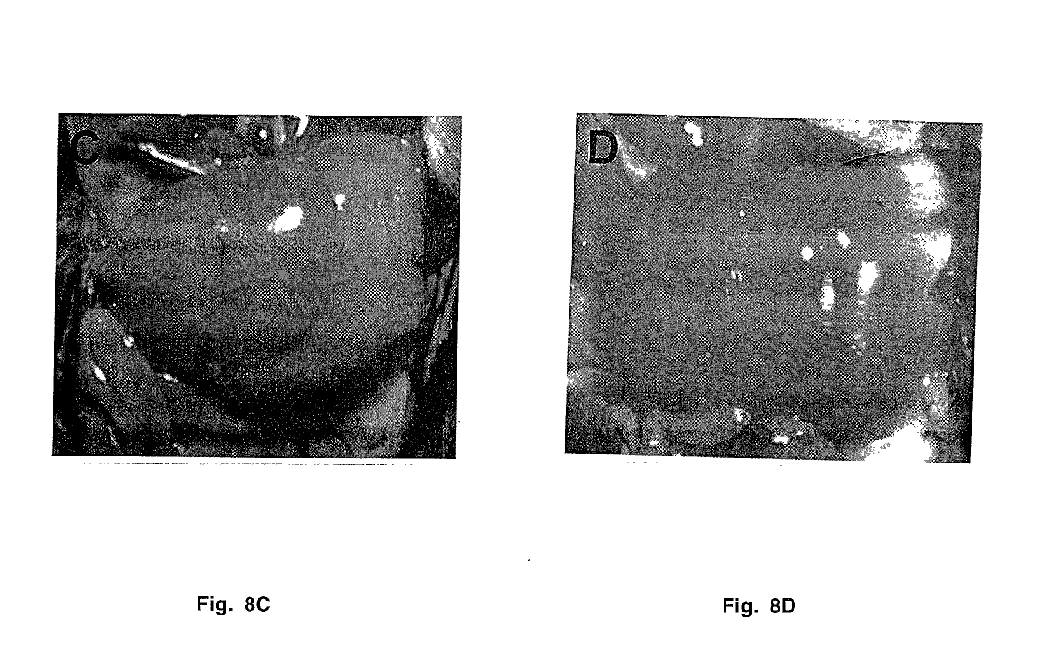

In the mice treated with AdCMV TK and GCV, the activity and food intake began to decrease from the day three, and 2 of the 5 mice of this group died on day 6. Macroscopically, in all surviving mice of this group, the color of the liver appeared yellowish, suggestive of fatty change (FIG. 8A). No remarkable abnormality was observed in other organs. Neither a decrease of activity nor macroscopic abnormality was observed in any other group including the group that received Adcox2LTK and GCV (FIGS. 8B-8D).

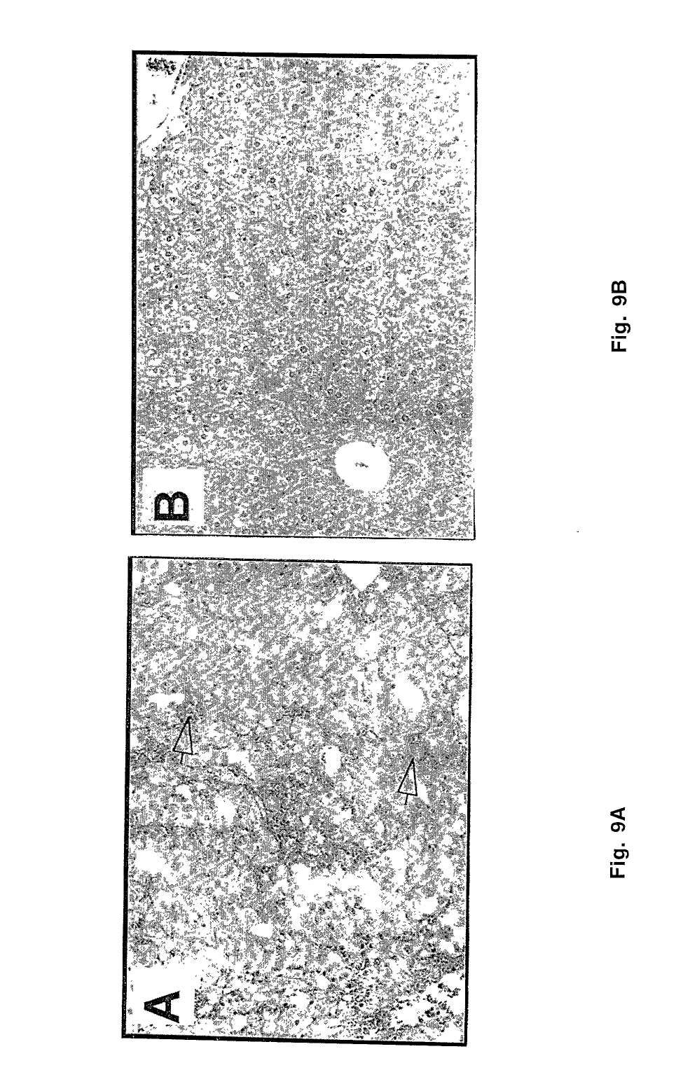

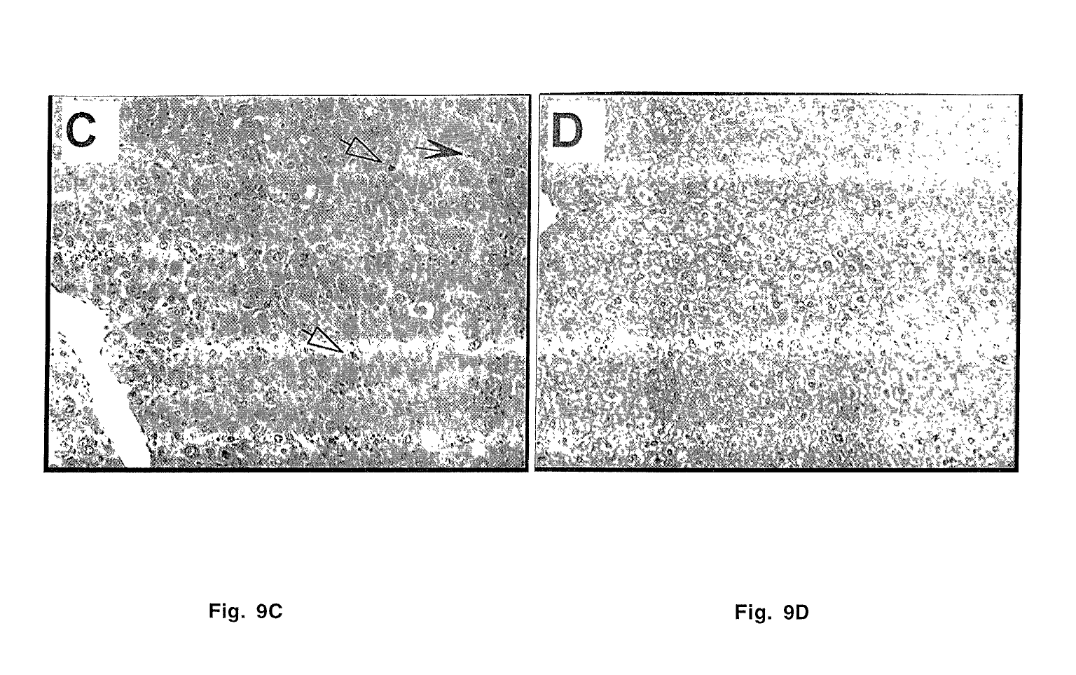

Microscopically, in the group treated with AdCMV TK and GCV, most of the hepatocytes showed extensive microvesicular fatty changes. There was also swelling of the individual hepatocytes, scattered individual cells undergoing necrosis, and marked nuclear pleomorphism of hepatocytes. This group also had marked congestion of the hepatic parenchyma (FIG. 9A). In the group with Adcox2L TK and GCV (FIG. 9B), the tinctorial pattern of hepatocytes was similar to the pattern seen in the untreated control (FIG. 9D). In this group, there were scattered individual necrotic cells and small areas of cellular dropout as well as focal areas of extramedullary hematopoiesis. In addition, this group showed scattered lymphocytes present in the hepatic parenchyma and the Kupffer cells were prominent. The livers of the group which received AdCMV TK without GCV (FIG. 9C) were remarkable for the extensive nuclear pleomorphism of the hepatocytes. There was an increase in mitosis, individual cell necrosis and apoptotic bodies. Though there was focal acute and lymphocytic inflammation scattered throughout the hepatic parenchyma, many necrotic cells were not surrounded by inflammatory cells. Overall, the patterns seen in this group was consistent with increased hepatic cellular injury and death followed by hepatic regeneration. The livers of the groups without virus administration appeared almost normal regardless of GCV administration (FIG. 9D).

No differences were observed in the histologic characteristics of the kidneys or the lungs among all groups. Spleens of groups which received AdCMV TK showed extensive extramedullary hematopoiesis and the demarcations between the white and red pulp were obscured. The group with Adcox2L TK and GCV showed some extramedullary hematopoiesis, although less extensive than that seen in the group with AdCMV TK.

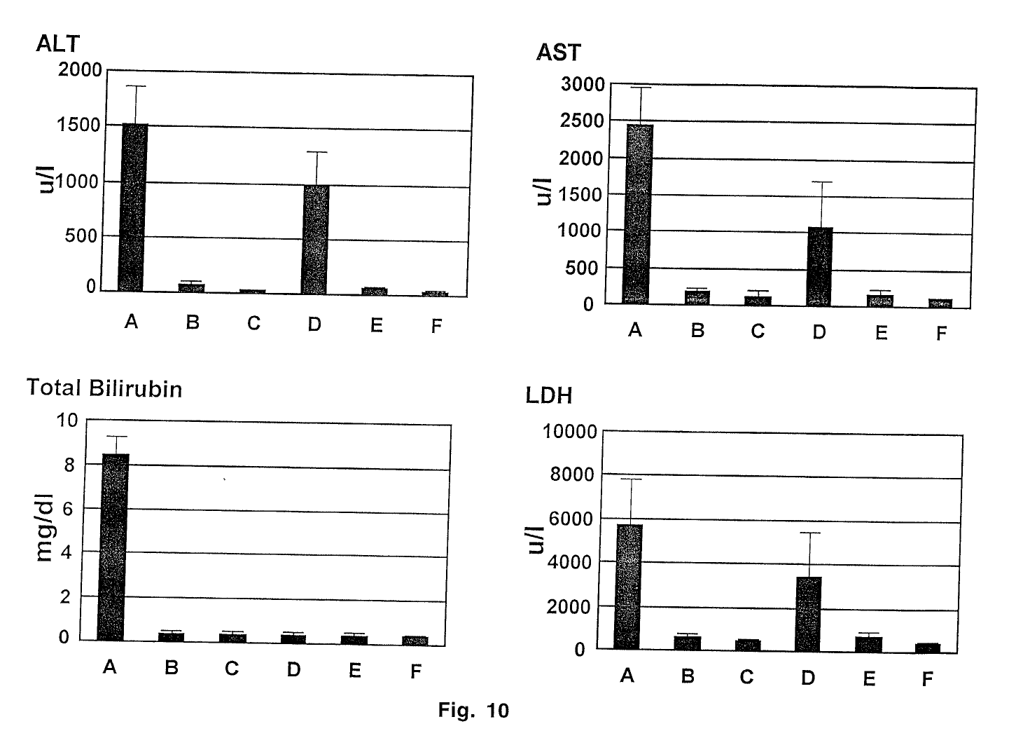

In the analysis of the serum samples, an elevation of the total bilirubin level was observed only in the group with AdCMV TK and GCV (FIG. 10A). In contrast, abnormality of ALT, AST and LDH appeared in the groups with AdCMV TK regardless of GCV administration (FIGS. 10B-D). Neither of the groups that received Adcox2L TK (with or without GCV) showed abnormality compared with the untreated group. The group that received only GCV (without virus) did not show abnormality. Taken together, the lethal hepatotoxicity with massive microvesicular fatty change and jaundice observed in AdCMV TK with GCV group was mitigated by substituting the CMV promoter with the cox-2 L promoter.

### Discussion

Adenoviral vectors have been widely used for cancer gene therapy [6]. However, in the context of suicide gene therapy, the expression of the effector gene in the liver due to vector hepatotropism is problematic as it can cause fatal liver dysfunction [32]. Especially in gastrointestinal cancers, where the most probable administration route is intravenous or transcatheter administration into the feeding artery of the tumor, the expression of a suicide gene in the liver is clearly one of the major obstacles to the clinical use of adenoviral vectors for suicide gene therapy.

To employ transcriptional targeting to overcome this issue, a promoter that is inactive in the liver but strongly active in gastrointestinal cancer cells was used. In the present study, the cyclooxygenase-2 promoter, which is expressed in many gastrointestinal cancers [17] but not expressed in most of the organs under normal conditions [15], was incorporated into an adenoviral vector construct and characterized in vitro and in vivo, and its feasibility for mitigating liver toxicity in suicide gene therapy was studied.

When the cyclooxygenase-2 promoters were incorporated in adenoviral vectors with unmodified fiber, the activity profile of in vitro experiments did not correlate with the cyclooxygenase-2 RNA status but rather showed correlation with infectivity (data not shown), mainly due to the apparent high luciferase activity observed in liver cancer cell lines. There were two possible explanations for these results: one is that higher infection efficiency of liver cancer-derived cells caused this high activity in these cells, the other is that the incorporation of the cyclooxygenase-2 promoter into an adenoviral vector made the promoter “leaky” as has been observed for other promoters [6-33-35].

To study this point, the RGD motif was incorporated into the H-I loop of the fiber knob region [30]. When using this infectivity-enhanced vector, with which the variation of infection efficiency was minimized from 20 fold to 20% (data not shown), the overall results showed correlation not with the infectivity but with cyclooxygenase-2 RNA status. This clearly indicated that the cyclooxygenase-2 promoter was working properly in the adenoviral construct and also suggested that the reason for the unexpected results with the adenovirus with unmodified fibers was not the leakiness of the promoter, but the variation of the infection efficiency.

In the experiments of tail vein administration of luciferase-expressing adenoviral vectors, the luciferase activity in the liver with the cyclooxygenase-2 M promoter was less than 1/400 of that with the CMV promoter and, moreover, that with cox-2 L promoter was even lower, less than 1/30000 of CMV promoter. On the other hand, the activities in the spleen with these three promoters were within the same order of magnitude. These results indicated that the cyclooxygenase-2 promoters are not generally weak but negative in the liver specifically. The cyclooxygenase-2 promoters, especially the cyclooxygenase-2 L promoter, had remarkable reduction of the transgene expression in liver and were potentially applicable for avoiding the liver toxicity caused by undesired transgene expression at that site.

Interestingly, although major positive control regions exist in the last 300 bp of these promoters, the activity of the cyclooxygenase-2 L promoter in the liver was much lower than the cyclooxygenase-2 M and this remarkable difference did not exist in the analysis using cell lines. This might suggest that a transcription suppressor sequence functioning in the in vivo liver existed between −1432 and −883. When the promoter was analyzed in the subcutaneous tumors, the cyclooxygenase-2 L promoter in cyclooxygenase-2 positive cells (MKN28 and MKN45) was very strong and comparable to the CMV promoter. Because the CMV promoter has been recognized as one of the strongest promoters in an adenoviral context [6-36], it may be understood that the cyclooxygenase-2 promoter is a relatively strong promoter in cyclooxygenase-2 positive tumors.

When the cyclooxygenase-2 promoters were incorporated into the HSV-tk expression vectors, the cyclooxygenase-2 promoter-driven vectors (Adcox2M TK and Adcox2L TK) showed strong cytocidal effects selectively on cyclooxygenase-2 positive cells, unlike the vector with the CMV promoter that killed all the cell lines regardless of cyclooxygenase-2 status. This confirmed that the cyclooxygenase-2 promoter was strong and selective enough to kill cyclooxygenase-2 positive cells in suicide gene therapy with HSV-tk and GCV. At the same time, because it is reported that the specificity of some promoters may vary with distinctive transgenes [37], it is important that selectivity of the promoter was confirmed with two different transgenes.

The in vitro and in vivo analyses indicated that the cyclooxygenase-2 L promoter was suitable for mitigation of the liver toxicity caused by the undesired transgene expression because its low activity in the liver and high activity in cyclooxygenase-2 positive tumors were well preserved in the adenoviral constructs. Thus, the in vivo toxicity of the cyclooxygenase-2 L promoter driven HSV-tk expression vector, in combination with GCV, was compared with that of the CMV promoter. When AdCMV TK was administered, GCV caused remarkable toxicity where 2 of the 5 mice of that group died at day 5. In this group, the liver of the surviving mice showed severe macroscopical and microscopical abnormality and the blood analysis showed hyperbilirubinemia and liver dysfunction. In contrast, in the group with the Adcox2LTK in combination with GCV administration, the liver was almost normal except for rare dropout of hepatocytes and extramedullary hematopoiesis. In addition, there was no significant abnormality in the blood analysis. This clearly indicated that the “liver off/tumor on” profile of the cyclooxygenase-2 promoter mitigated the fatal liver toxicity. Interestingly, even without GCV administration, the liver receiving AdCMV TK showed extensive damage without microvesicular fatty change. Because the histological localization of the liver damage in this group was independent from lymphocyte infiltration, it suggested that this liver damage was caused by direct toxicity of HSV-tk expression in the liver, independently from cellular immunity.

Looking for the method to overcome the safety issue, some tumor specific promoters have been employed to drive HSV-tk expression in adenoviral vectors. In the context of suicide gene therapy of Wilms' tumor and neuroblastoma, the midkine promoter is reported to be very beneficial for the mitigation of liver toxicity [38]. In the field of gastrointestinal cancers, though the carcino-embryonic antigen promoter is reported to show a useful profile in terms of low activity in the liver, the activity of this promoter on the 20 positive cell lines is relatively low and required a 20 fold dose to obtain transgene expression of a comparable level to that of the CMV promoter [39]. Because the fatal toxicity in the dose escalation in a clinical study of ornithine transcarbamylase deficiency suggesting the possibility of toxicity of adenoviral vectors at high dose [40], such dose increase as a means to overcome the weakness of the promoter is not practical for clinical use. Thus, selective and strong promoters in gastrointestinal cancers are required to make gene therapy feasible and, in this context, the cyclooxygenase-2 promoter appears to be especially promising.

In the present study, the cyclooxygenase-2 promoter was characterized in an adenoviral construct and reduced undesired transgene expression in the liver. In suicide gene therapy with a n HSV-tk expressing adenoviral vector and GCV administration, this promoter mitigates the hepatotoxicity after systemic administration. This indicates that the cyclooxygenase-2 promoter can be very beneficial for gene therapy of intrahepatic lesions by transcatheter intraarterial administration or intratumoral injection, where the transgene expression in the background liver can cause safety problems [12]. To make gene therapy not only feasible but also clinically useful, strategies to ensure key safety endpoints are required. In this regard, cyclooxygenase-2 promoters are useful to reduce the adverse effect caused by the undesired transgene expression in the liver and have widespread applicability for many kinds of cox-2 positive cancers [41-44].

### EXAMPLE 9

### Cyclooxygenase-2 Promoter Regions In Adenoviral Context Have High Activity In Pancreatic Carcinoma

The established human pancreatic carcinoma cell lines (BxPC-3, Capan-1, Hs766-T and MIA PaCa-2; >20 passages) were purchased from Boehringer Ingelheim, Belgium. The low passage (primary) human pancreatic carcinoma cells (p6.3 and p10.5; <5 passages) were obtained from Dr. E. Jaffee, Johns Hopkins University School of Medicine, Baltimore. KATO III cells (gastric carcinoma) were purchased from the American Type Culture Collection (Manassas, Va.). KATO III cells were cultured with RPMI 1640 (Mediatech, Herndon, Va.) with 20% Fetal Bovine Serum (FBS; Summit Biotechnology, Ft. Collins, Colo.), 1% L-glutamine and 1% penicillin/streptomycin (Life Technologies Inc., Rockville, Md.) at 37° C. in 5% carbon dioxide atmosphere at 95% humidity. The other cells were cultured at the same conditions, but in this case Dulbecco's minimal essential medium (DMEM) (Mediatech, Herndon, Va.) with 10% FBS is used.

The E1; E3-deleted adenovirus vector expressing the firefly luciferase gene from the cytomegalovirus (CMV) immediate early promoter, AdCMVluc, was obtained from Dr. R. Gerard (University of Leuven, Leuven, Belgium). AdCMV-HSV-Tk, expressing the herpes simplex virus thymidine kinase gene, was constructed as described earlier [45]. AdCMV GFP was obtained from Dr. Parameshwar, Gene Therapy Center, University of Alabama, Birmingham. The recombinant Ad vectors AdCOX-2M Luc and AdCOX-2L Luc as well as AdCOX-2M Tk and AdCOX-2L Tk, under the control of human COX-2M and COX-2L promoter regions, respectively, were constructed using the “AdEasy” method reported previously [27].

Briefly, the COX-2M and COX-2L [38] with the luciferase gene (pGL3 basic vector, Promega) and the COX-2M and COX-2L promoters with the Tk gene [45] were inserted into a multiple cloning site in the pShuttle vector. The resultant plasmid was linearized with Pme1 digestion and subsequently cotransfected into E.coli BJ5183 with pAdEasy-1 adenoviral backbone plasmid. After selection of recombinants in these bacteria, the recombinant of interest were grown up and linearized with Pac1 digestion and transfected into 293 cells to generate AdCOX-2M Luc, AdCOX-2L Luc, AdCOX-2M Tk, and AdCOX-2L Tk. The recombinant adenoviruses were propagated in 293 cells, purified by double CsCl density centrifugation and virus titers were determined by plaque assay, as described.

To assess adenovirus-infection efficiency 5×10⁴ of each of the tumor cells per well were plated in 24-well plates and allowed to adhere overnight. The next day the cells were infected with AdCOX-2M Luc, AdCOX-2L Luc, AdMKLuc or AdCMVLuc at a multiplicity of infection (MOI) of 1, 10 and 100 per cell in phosphate buffered saline (PBS) for 1 h at 37° C. Cell were incubated in PBS without virus as a control. The virus was removed and the cells were incubated for 48 hrs in complete media. The infected cells were harvested and treated with 100 μl lysis buffer. A luciferase assay (Luciferase Assay System, Promega Corp., Madison, Wis.) and a Berthold luminometer (Lumat, Wallc Inc.) were used for the evaluation of luciferase activities of Ad infected cells. Luciferase activities were normalized by the protein concentration in the cell lysate using the BCA Protein Assay procedure (Pierce, San Fransisco, Calif.) as described by the manufacturer.

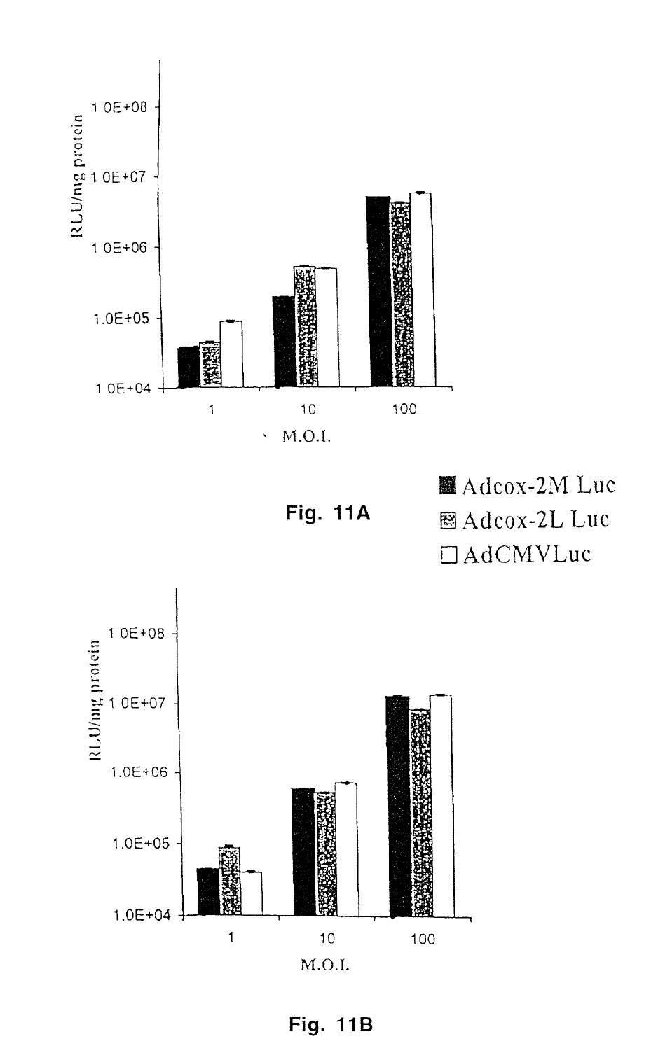

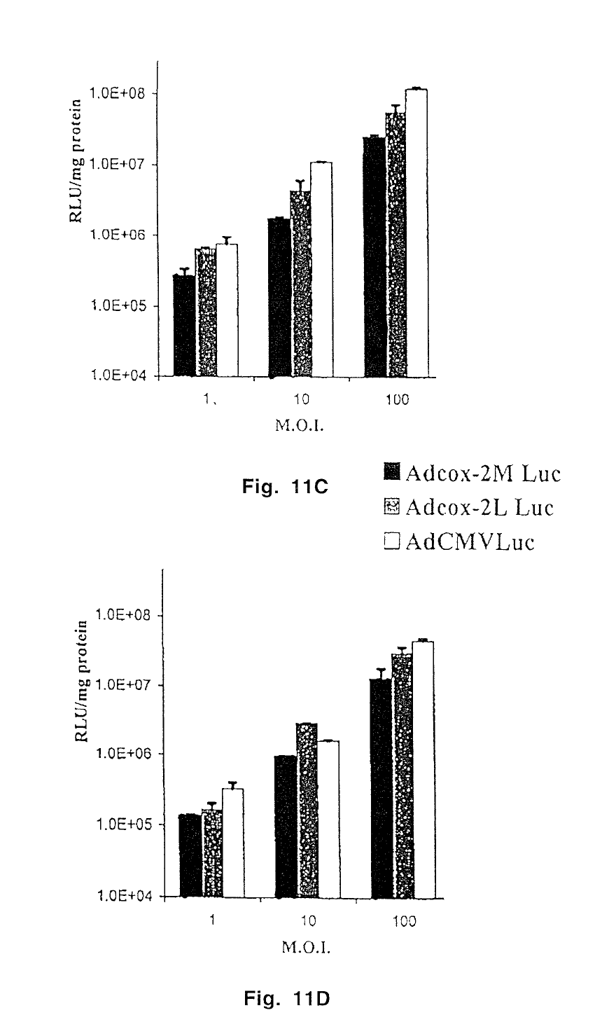

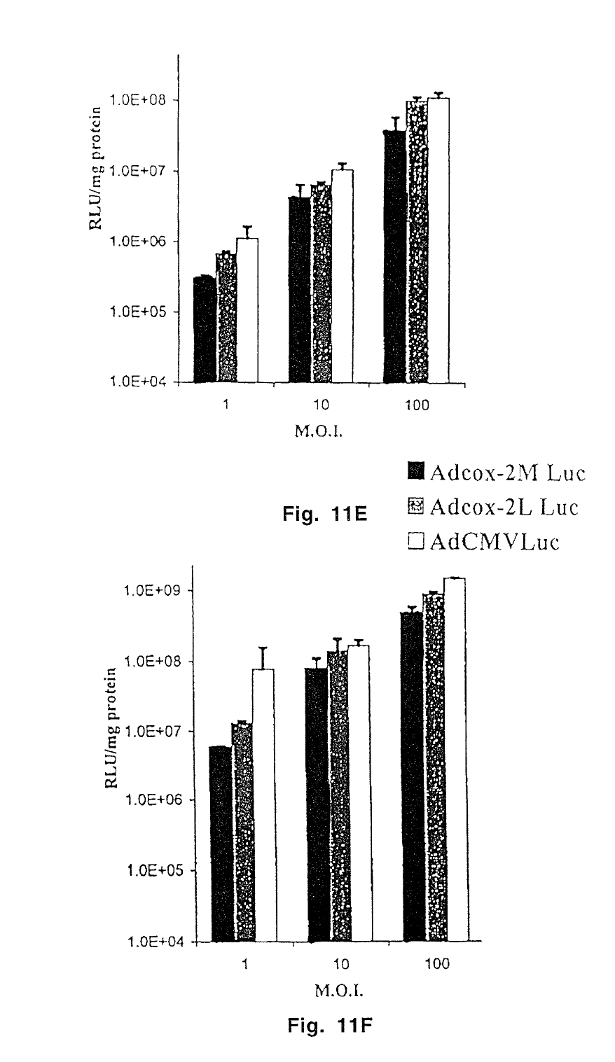

Since the cyclooxygenase-2 gene expression has been shown to be upregulated in human pancreatic carcinoma cells [47], whether cyclooxygenase-2 promoter regions in the adenoviral context would manifest high transcriptional efficiency was investigated in primary and in established pancreatic carcinoma cells. Although three major control regions of the cyclooxygenase-2 promoter (NF-κB, NFIL-6 and CRE) exist within 300 bps from the transcription initiation site [28, 29], two longer control regions (COX-2M (−883 bp to +59 bp) and COX-2L (−1432 bp to +59 bp)) were used to obtain more fidelity. In a reporter gene experiment with firefly luciferase, primary and established human pancreatic carcinoma cells were infected with AdCOX-2M Luc, AdCOX-2L Luc or AdCMVLuc at MOIs of 1, 10 and 100 (FIG. 11). In the primary pancreatic carcinoma cells (p6.3 and p10.5) luciferase activity induced by both AdCOX-2M as well as AdCOX-2L revealed high activity, approximately the same activity as induced by the AdCMVLuc infection.

Interestingly, virtually no difference of this high luciferase activity between the two COX-2 promoter constructs (COX-2M and COX-2L) was observed (FIGS. 11A and 11B). In the four established pancreatic carcinoma cell lines (BxPC-3, Capan-1, Hs 766T and MIA PaCa-2) the luciferase activity induced by AdCOX-2M and AdCOX-2L also showed this high activity, but in this case AdCOX-2L has approximately 50% to 80% of the luciferase activity induced by AdCMVLuc infection, while AdCOX-2M showed 20% to 30% of the activity induced by AdCMVLuc infection (FIGS. 11C-11F). As a negative control gastric cancer cells KATO III were infected with AdCOX-2M and AdCOX-2L revealing less than 1% luciferase activity as compared with AdCMVLuc (results not shown).

In comparison with results of tumor specific promoters published previously [48], the luciferase activity induced by the COX-2 promoter regions in human pancreatic tumors as depicted in FIG. 11 can be regarded as high and, therefore, the use of especially the COX-2L promoter region looks promising for tumor-specific gene therapy approaches in pancreatic carcinoma.

### EXAMPLE 10

### Effect Of Cox-2 Promoter In Combination With Tk And GCV In Pancreatic Carcinoma Cells

Four pancreatic carcinoma cell lines as well as a control tumor cell line (gastric cancer, KATO III) were plated in 96 well plates in triplicate at a density of 3000 cells per well. After overnight culture, cells were infected with AdCOX-2M Tk, AdCOX-2L Tk, AdCMVTk and AdCMVGFP as a control at an MOI of 500 during 5 hrs. The viral infection was followed by medium replacement including various concentrations of ganciclovir (GCV) ranging from 0 to 10000 μM. The number of surviving cells was determined by a 3-(4,5 -dimethylthiazol-2-yl)-2,5-diphenyltetrazolium bromide (MTT) assay (CellTiter 96 Aqueous Non-Radioactive Cell Proliferation Assay, Promega) after 5 days exposure of GCV using an automated E max spectrophotometric plate reader (Molecular Device Corp., Sunnyval, Calif.). The depicted results are the mean of triplicate assays and the standard deviation was assessed on basis of the three determinations.

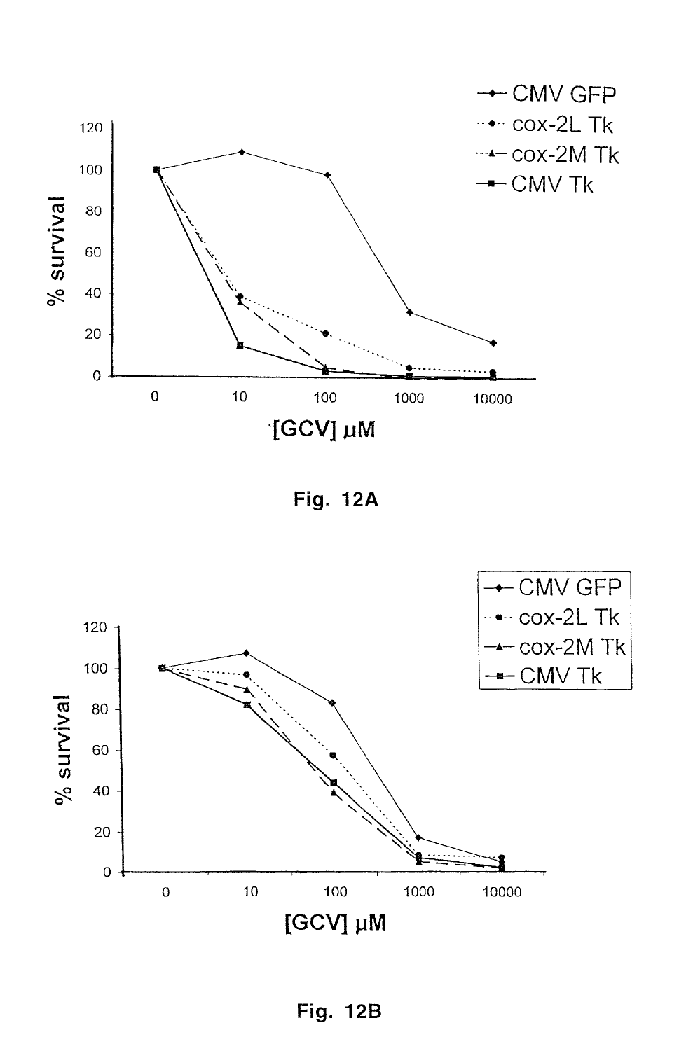

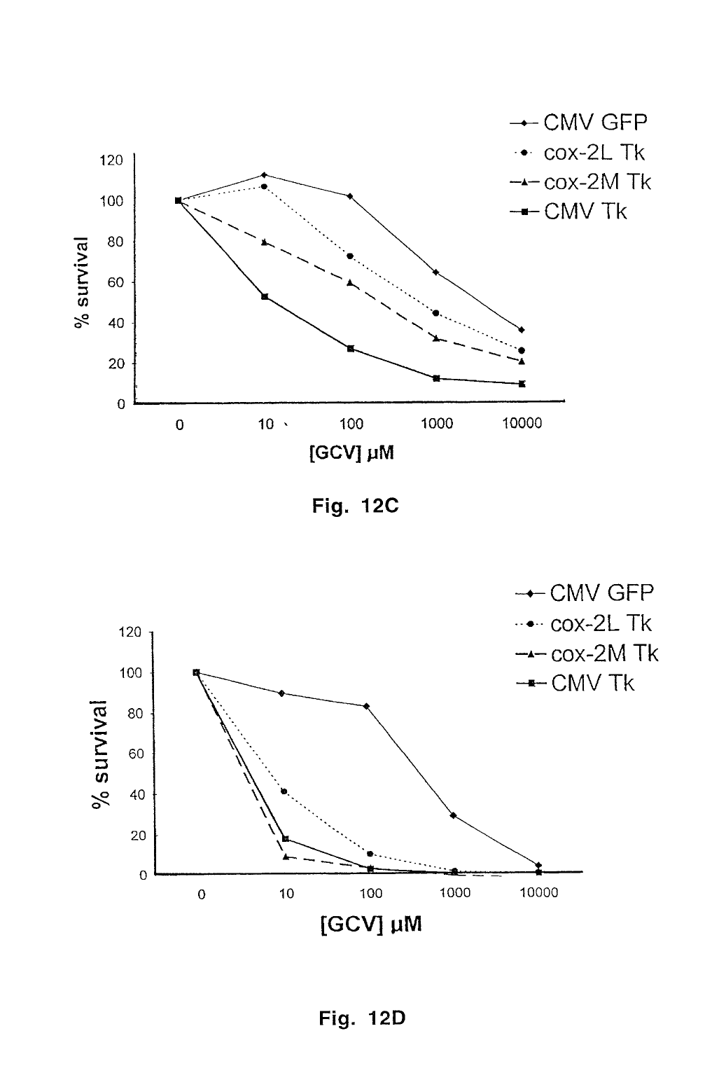

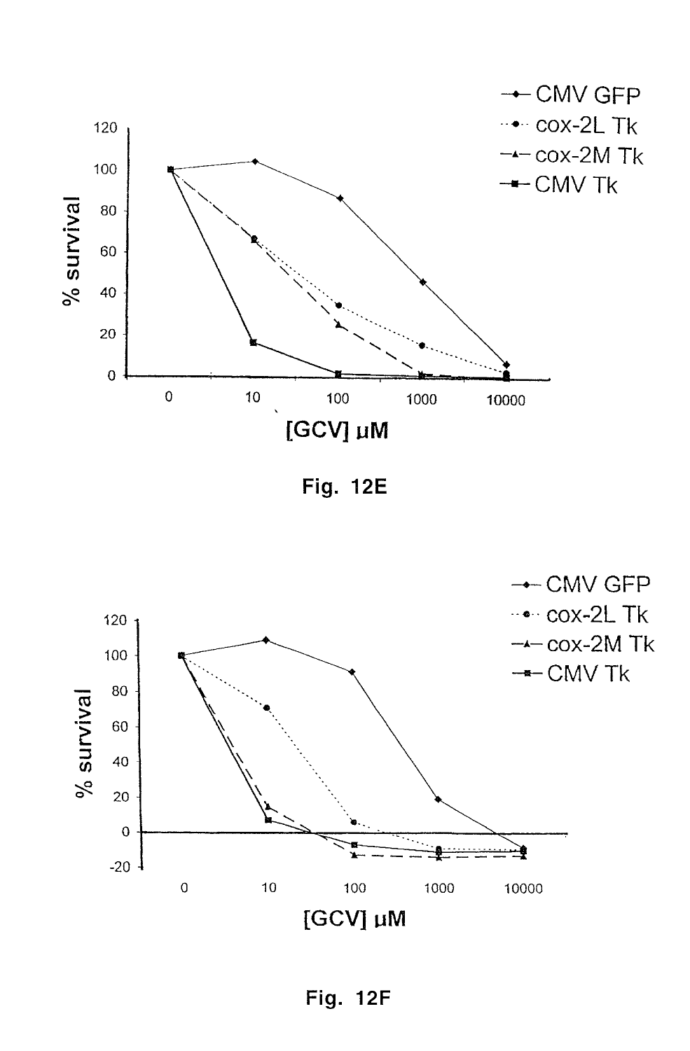

As shown in FIG. 11, both AdCOX-2M Tk as well a s AdCOX-2L Tk virus vectors successfully induced GCV sensitivity in the BxPC-3 and MIA PaCa-2 pancreatic carcinoma cell lines with IC50 values (the GCV concentration at which 50% cell survival is seen) comparable to those obtained with the AdCMVTk virus (FIGS. 12A and 12D). For the pancreatic carcinoma cell line Hs766-T and the gastric cancer cell line KATO III the induced GCV sensitivity by the AdCOX-2M Tk or AdCOX-2L Tk viruses was much less than seen with AdCMV Tk (FIGS. 12C and 12E). Although the luciferase study revealed that both the COX-2M and COX-2L promoter activity was comparable with the CMV promoter activity in the Capan-1 cells (FIG. 11D), the GCV sensitivity caused by AdCOX-2M/2L Tk/GCV was very limited, which might be caused by the relative resistance of the Capan-1 cells for GCV (FIG. 12B). The data revealed that AdCOX-2M/2L Tk induced marked cell-death in response to GCV in two out of four pancreatic carcinoma cell lines, suggesting that the cyclooxygenase-2 promoter in combination with Tk and GCV might be a promising candidate for tumor specific suicide gene therapy of human pancreatic tumors.

Human pancreatic carcinoma cells have been shown to be relatively resistant to adenovirus gene transfer, but the use of both epidermal growth factor receptor (EGFR) targeted Ad vectors as well as integrin targeted (i.e. RGD (Arg-Gly-Asp)-modified) Ad-vectors have demonstrated enhanced gene transfer to both primary as well as established pancreatic carcinoma cells [30, 49]. To further improve the selectivity of expressing therapeutic genes in pancreatic cancer, this study showed for the first time that two cyclooxygenase-2 promoter regions in the adenoviral backbone revealed tumor specific expression in both primary as well as established human pancreatic carcinoma cells. Furthermore, experiments with the Tk gene driven by the cyclooxygenase-2 promoter regions clearly indicate that this promoter may be an ideal candidate for tumor-specific suicide gene therapy of pancreatic cancer. Also, the low level of expression of the midkine promoter in the liver, combined with the lack of liver dysfunction upon AdMKTk/GCV administration, as shown in a previous study by Adachi et al. [38], will prevent liver toxicity reported for the AdCMV-Tk/GCV approach [11]. Furthermore, in a recent study Yip-Schneider et al. revealed that the cyclooxygenase-2 protein expression in primary human pancreatic adenocarcinoma was found to be significantly elevated than in corresponding normal pancreas tissue [50]. Relatively high levels of expression of cyclooxygenase-2 in pancreatic tumors will have a significantly enhanced therapeutic window compared to the use of viral promoters such as CMV. From the results of this study it appeared that the COX-2L promoter region revealed a more profound tumor-specific activity in, pancreatic carcinoma than the COX-2M promoter, which fits nicely with the observed reduced expression of this promoter region in normal human liver, as has been shown for the midkine-promoter in an earlier study [38].

The finding that the cyclooxygenase-2 promoter regions revealed a luciferase activity comparable as induced by the CMV promoter in both primary and in established pancreatic carcinoma cells, is of importance because most tumor-specific promoters that have been proposed for use in gene therapy vectors previously exhibit levels of activity that are much lower than viral promoters [48]. This has lead to the development of amplification strategies to enhance the efficiency of these specific but weak promoters. Furthermore, a number of candidate tumor-specific promoters have lost a significant part of their specificity when cloned in the adenoviral backbone, probably due to cis- or trans-acting enhancing elements in the genome of the Ad vector. In this study, however, both cyclooxygenase-2 promoters retain their activity and specificity in the Ad context.

In summary, this study clearly demonstrates that the cyclooxygenase-2 promoters are two promising candidate tumor-specific promoters, since they reveal low hepatic activity and toxicity, they show high tumor activity in both primary- and established human pancreatic carcinoma cells and they reveal fidelity in the adenoviral backbone. Combined with the targeted Ad vectors as described previously [30, 49], the COX-2 promoters may be ideal candidate tumor-specific promoters for cancer gene therapy of pancreatic tumors in humans.

The following references were cited herein:

1. Levin B. Neoplasm of the Large and Small Intestine. In: Bennett J C and Plum F, eds. Textbook of Medicine. Volume 1. 20th ed. Philadelphia: Saunders, 1996:721-729.

2. Kurtz and Winawer. Neoplasm of the Stomach. In: Bennett J C and Plum F, eds. Textbook of Medicine. Volume 2. 20th ed. Philadelphia: Saunders, 1996:676-680.

3. Vile and Hart, Cancer Res 53:3860-3864, 1993.

4. Moolten and Wells, J Natl Cancer Inst 82:297-300, 1990.

5. Culver et al., Science 256:1550-1552, 1992.

6. Hitt et al., Adv Pharmacol 40:137-206, 1997.

7. Kanai et al., Hepatology 23:1359-1368, 1996.

8. Ohashi et al., Jpn J Cancer Res 89:457-462, 1998.

9. Huard et al., Gene Ther 2:107-115, 1995.

10. Reynolds et al., Gene Ther 6:1336-1339, 1999.

11. van der Eb et al., Gene Ther 5:451-458, 1998.

12. Bilbao et al., Cancer Gene Ther 7:657-662, 2000.

13. Krasnykh et al., Receptor-Specific Targeting of Adenovirus Vector via Genetic Replacement of the Fiber Protein. (Submitted).

14. Roelvink et al., Science 286:1568-1571, 1999.

15. Dubois et al., Faseb J 12:1063-1073, 1998.

16. Tippetts et al., Mol Cell Biol 8:4570-4572, 1988.

17. Williams et al., Ann N Y Acad Sci 889:72-83, 1999.

18. Tsujii et al., Cell 83:493-501, 1995.

19. Eberhart et al., Gastroenterology 107:1183-1188, 1994.

20. Sano et al., Cancer Res 55:3785-3789, 1995.

21. Kutchera et al., Proc Natl Acad Sci U S A 93:4816-4820, 1996.

22. Kargman et al., Cancer Res 55:2556-2559, 1995.

23. Su et al., Science 256:668-670, 1992.

24. Singh et al., Cancer Res 57:3465-3470, 1997.

25. Williams et al., Gastroenterology 111:1134-1140, 1996.

26. Ristimaki et al., Cancer Res 57:1276-1280, 1997.

27. He et al., Proc Natl Acad Sci USA 95:2509-2514, 1998.

28. Inoue et al., J Biol Chem 270:24965-24971, 1995.

29. Inoue et al., FEBS Lett 350:51-54, 1994.

30. Dmitriev et al., J Virol 72:9706-9713, 1998.

31. Krasnykh et al., J Virol 72:1844-1852, 1998.

32. Brand et al., Cancer Gene Ther 4:9-16, 1997.

33. Steinwaerder and Lieber, Gene Ther 7:556-567, 2000.

34. Vassaux et al., Gene Ther 6:1192-1197, 1999.

35. Zhang and Russell, Cancer and Metastasis Review 15:385-401, 1996.

36. Addison et al., J Gen Virol 78:1653-1661, 1997.

37. Kurachi et al., Science 285:739-743, 1999.

38. Adachi et al., Cancer Res 60:4305-4310, 2000.

39. Brand et al., Gene Ther 5:1363-1371, 1998.

40. Lehrman, Nature 401:517-518, 1999.

41. Wolff et al., Cancer Res 58:4997-5001, 1998.

42. Molina et al., Cancer Res 59:4356-4362, 1999.

43. Shirvani et al., Gastroenterology 118:487-496, 2000.

44. Yoshimura et al., Cancer 89:589-596, 2000.

45. Garver et al., Gene Ther. 1: 46-50, 1994.

46. Adachi et al., Oncogene 13: 2197-2203, 1996.

47. Tucker et al., Cancer Res. 59: 987-990, 1999.

48. Ohashi et al., Jap. J. Cancer Res. 89: 457-462, 1998.

49. Dmitriev et al., J. Virol. 74: 6875-6884, 2000.

50. Segawa et al., Cancer Res. 58: 2282-2287, 1998.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

One skilled in the art will appreciate readily that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. An adenoviral vector for the selective expression of toxin gene in cancer cell, comprising a toxin gene operably linked to a promoter of a gene with undetectable expression in liver, wherein the expression of said toxin gene is reduced in liver cells.

2. The adenoviral vector of claim 1, wherein said promoter is cyclooxygenase-2 promoter.

3. The adenoviral vector of claim 2, wherein said cyclooxygenase-2 promoter is selected from the group consisting of cyclooxygenase-2 L (−1432/+59) and cyclooxygenase-2 M (−833/+59).

4. The adenoviral vector of claim 1, wherein said cancer cell is selected from the group consisting of gastrointestinal cancer cell and pancreatic cancer cell.

5. The adenoviral vector of claim 1, wherein said toxin gene is selected from the group consisting of the herpes simplex virus thymidine kinase gene, the cytosine deaminase gene and the purine nucleoside phosphorylase gene.

6. The adenoviral vector of claim 1, further comprises a RGD motif in the HI loop of the adenovirus fiber protein.

7. A method of killing tumor cells with reduced liver toxicity in an individual, comprising the step of:
administering a therapeutically effective amount of adenoviral vector comprising a toxin gene operably linked to a promoter of a gene with undetectable expression in liver, wherein expression of said toxin gene is reduced in liver cells and expression of said toxin gene in tumor cells results in killing of said tumor cells.

8. The method of claim 7, wherein said administering is by intravenous injection.

9. The method of claim 7, wherein said tumor cells are selected from the group consisting of gastrointestinal cancer cells and pancreatic cancer cells.

10. The method of claim 7, wherein said promoter is cyclooxygenase-2 promoter.

11. The method of claim 10, wherein said cyclooxygenase-2 promoter is selected from the group consisting of cyclooxygenase-2 L (−1432/+59) and cyclooxygenase-2 M (−833/+59).

12. The method of claim 7, wherein said toxin gene is selected from the group consisting of the herpes simplex virus thymidine kinase gene, the cytosine deaminase gene and the purine nucleoside phosphorylase gene.

13. The method of claim 7, wherein said toxin gene is the herpes simplex virus thymidine kinase gene, and further comprises a step of treating said individual with ganciclovir.

14. The method of claim 7, wherein said adenoviral vector further comprises a RGD motif in the HI loop of the adenovirus fiber protein.
